(19) United States
(12) Patent Application Publication
(10) Pub. No.: US 2013/0202586 A1
(43) Pub. Date: Aug. 8, 2013

(54) STEM CELL THERAPY USING INHIBITORS OF LYSOPHOSPHATIDIC ACID

(71) Applicant: Roger A. SABBADINI, Lakeside, CA (US)

(72) Inventor: Roger A. SABBADINI, Lakeside, CA (US)

(21) Appl. No.: 13/570,714

(22) Filed: Aug. 9, 2012

Related U.S. Application Data
(60) Provisional application No. 61/521,714, filed on Aug. 9, 2011.

(51) Int. Cl. A61K 39/395; C12N 5/071

(52) U.S. Cl. CPC A61K 39/39533; C12N 5/0602
USPC 424/133.1; 424/172.1; 435/377

(57) ABSTRACT

Methods are provided for stem cell therapy using inhibitors of lysophosphatidic acid (LPA). Inhibition of LPA may be direct or indirect; particularly preferred direct inhibitors of LPA are antibodies to LPA, including humanized monoclonal antibodies to LPA. Such inhibitors are used in combination with stem cells for the treatment of injuries, diseases, or conditions amenable to treatment by stem cell therapy.

### TECHNICAL FIELD

The present invention relates to methods of stem cell therapy using inhibitors of lysophosphatidic acid (LPA). Such inhibitors may inhibit LPA directly or indirectly; preferred inhibitors of LPA are antibodies, preferably humanized monoclonal antibodies, to LPA.

### BACKGROUND OF THE INVENTION

1. Introduction

The following description includes information that may be useful in understanding the present invention. It is not an admission that any such information is prior art, or relevant, to the presently claimed inventions, or that any publication specifically or implicitly referenced is prior art or even particularly relevant to the presently claimed invention.

2. Background

A. Stem Cells and Stem Cell Therapy

Stem cells are undifferentiated cells capable of either renewing their own cell population or differentiating into specialized, differentiated cells. Types of stem cells include embryonic stem cells (ESCs), adult stem cells (ASCs) and umbilical cord stem cells. In addition, the generation of induced pluripotent stem cells (iPSCs) from the somatic cells of humans (Takahashi and Yamanaka, (2006) Cell 126:663-676) has added to the tools available for stem cell therapy. Like ESCs, iPSCs have the ability to proliferate endlessly and yet have the potential to differentiate into derivatives of all three germ layers.

Stem cell transplantation is currently in clinical trials in several human neurodegenerative disorders such as Parkinson's Disease (PD), Huntington's Disease (HD), amyotrophic lateral sclerosis (ALS), syringomyelia, and others. Examples of diseases or conditions currently or potentially suitable for stem cell therapy include bone diseases and conditions, including joint defects and injuries; neuromuscular diseases and conditions such as muscle damage, amyotrophic lateral sclerosis (ALS) and muscular dystrophy; cardiac diseases or conditions including myocardial infarct and heart failure; ischemic conditions including those of the heart; pancreatic disease or conditions including diabetes; neurological disease or conditions including traumatic brain injury, brain, or spinal cord hemorrhage, spinal cord injury, stroke, and neurodegenerative disease, including Parkinson's disease, Alzheimer's disease, Huntington's disease, and neurodegenerative disorders of the gastrointestinal tract causing motility disorder; liver disease; pulmonary disorders; and diseases and conditions of the skin, hair, and nails such as radiation injury, wounds, and baldness.

Stem cell therapy can also be a useful alternative in situations where there is a lack of availability of organs (e.g. liver) for organ transplantation. To date, only limited clinical success has been seen in stem cell transplantation.

The many challenges to successful stem cell therapy include (1) improving the homing and transdifferentiation of transplanted stem cells and (2) increasing the performance of stem cells once they have taken up residence in the target tissue. In tissue injury, changes in the microenvironment of the injured tissue may not favor survival of the transplanted cells (Richardson, et al., 2010 J. Neurosurg. 112: 1125-1138). For example, inflammatory cascades, fibrosis, macrophage and neutrophil activity, cytokine release, immune responses, haemorrhage, etc, may cause a ‘hostile environment’ for both seeded and resident stem cell activity. In the case of neurotrauma, these processes can contribute to continued neuronal cell death up to 12 months after the injury to the human brain (Williams, et al., 2001 Acta Neuropathol 102: 581-590). It has been suggested that in tissues such as the heart, liver, and brain, there is a continued process of cell loss and regeneration, and that tissue degeneration (i.e., cell loss) during aging is, in part, a failure of the tissue to regenerate. Thus, it is important to indentify, and possibly neutralize, key players in the tissue that can impede stem cell activity. It has been suggested that the injury microenvironment needs to guide this differentiation.

While most investigators have focused on protein signalling molecules such as cytokines, bioactive lipid mediators may also be dysregulated in the hostile microenvironment in which stem cells must operate, particularly in cases where stem cells must undergo transdifferentiation as, often times undifferentiated cells are transplanted. Lysophosphatidic acid (LPA) is an inflammatory lipid that acts on various stem cells, including neural stem/progenitor cell (NS/PC). While more is currently known about the role of LPA in neural stem cell differentiation, it is believed that LPA may play a similar role in differentiation of other types of stem cells, and thus reduction of LPA activity or effective concentration is believed to fill a long-felt need in the area of stem cell therapy, by enhancing the survival, homing, engraftment and differentiation of stem cells.

B. LPA and Other Lysolipids

Lysolipids are low molecular weight lipids that contain a polar head group and a single hydrocarbon backbone, due to the absence of an acyl group at one or both possible positions of acylation. Relative to the polar head group at sn-3, the hydrocarbon chain can be at the sn-2 and/or sn-1 position(s) (the term “lyso,” which originally related to hemolysis, has been redefined by IUPAC to refer to deacylation). These lipids are representative of signaling, bioactive lipids, and their biologic and medical importance highlight what can be achieved by targeting lipid signaling molecules for therapeutic, diagnostic/prognostic, or research purposes (Gardell, et al. (2006), Trends in Molecular Medicine, vol 12: 65-75). Two particular examples of medically important lysolipids are LPA (glycerol backbone) and S1P (sphingoid backbone). Other lysolipids include sphingosine, lysophosphatidylcholine (LPC), sphingosylphosphorylcholine (lysosphingomyelin), ceramide, ceramide-1-phosphate, sphinganine (dihydrosphingosine), dihydrosphingosine-1-phosphate and N-acetyl-ceramide-1-phosphate. In contrast, the plasmalogens, which contain an O-alkyl (—O—CH₂—) or O-alkenyl ether at the C-1 (sn1) and an acyl at C-2, are excluded from the lysolipid genus. The structures of selected LPAs, S1P, and dihydro S1P are presented below.

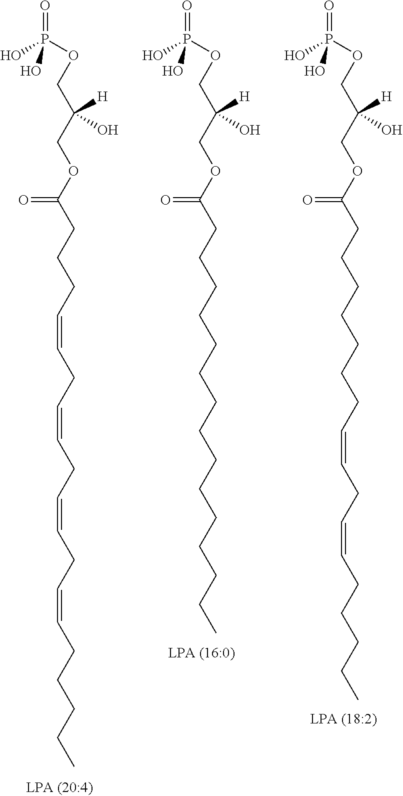

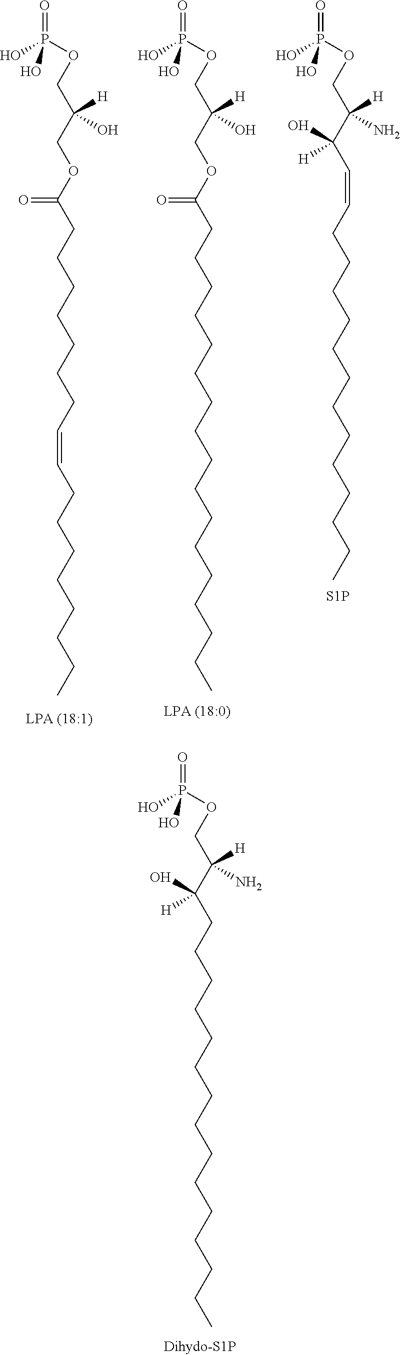

LPA is not a single molecular entity but a collection of endogenous structural variants with fatty acids of varied lengths and degrees of saturation (Fujiwara, et al. (2005), J Biol Chem, vol. 280: 35038-35050). The structural backbone of the LPAs is derived from glycerol-based phospholipids such as phosphatidylcholine (PC) or phosphatidic acid (PA). In the case of lysosphingolipids such as SIP, the fatty acid of the ceramide backbone at sn-2 is missing. The structural backbone of S1P, dihydro S1P (DHS1P) and sphingosylphosphorylcholine (SPC) is based on sphingosine, which is derived from sphingomyelin.

LPA and S1P are bioactive lipids (signaling lipids) that regulate various cellular signaling pathways by binding to the same class of multiple transmembrane domain G protein-coupled (GPCR) receptors (Chun and Rosen (2006), Current Pharm Des, vol. 12: 161-171, and Moolenaar, W H (1999), Experimental Cell Research, vol. 253: 230-238). The S1P receptors are designated as S1P₁, S1P₂, S1P₃, S1P₄ and S1P₅ (formerly EDG-1, EDG-5/AGR16, EDG-3, EDG-6 and EDG-8) and the LPA receptors designated as LPA₁, LPA₂, LPA₃ (formerly, EDG-2, EDG-4, and EDG-7). A fourth LPA receptor of this family has been identified for LPA (LPA₄), and other putative receptors for these lysophospholipids have also been reported.

LPA have long been known as precursors of phospholipid biosynthesis in both eukaryotic and prokaryotic cells, but LPA have emerged only recently as signaling molecules that are rapidly produced and released by activated cells, notably platelets, to influence target cells by acting on specific cell-surface receptor (see, e.g., Moolenaar, et al. (2004), BioEssays, vol. 26: 870-881, and van Leewen et al. (2003), Biochem Soc Trans, vol 31: 1209-1212). Besides being synthesized and processed to more complex phospholipids in the endoplasmic reticulum, LPA can be generated through the hydrolysis of pre-existing phospholipids following cell activation; for example, the sn-2 position is commonly missing a fatty acid residue due to deacylation, leaving only the sn-1 hydroxyl esterified to a fatty acid. Moreover, a key enzyme in the production of LPA, autotoxin (lysoPLD/NPP₂), may be the product of an oncogene, as many tumor types up-regulate autotoxin (Brindley, D. (2004), J Cell Biochem, vol. 92: 900-12). The concentrations of LPA in human plasma and serum have been reported, including determinations made using a sensitive and specific LC/MS procedure (Baker, et al. (2001), Anal Biochem, vol 292: 287-295). For example, in freshly prepared human serum allowed to sit at 25° C. for one hour, LPA concentrations have been estimated to be approximately 1.2 mM, with the LPA analogs 16:0, 18:1, 18:2, and 20:4 being the predominant species. Similarly, in freshly prepared human plasma allowed to sit at 25° C. for one hour, LPA concentrations have been estimated to be approximately 0.7 mM, with 18:1 and 18:2 LPA being the predominant species.

LPA influences a wide range of biological responses, ranging from induction of cell proliferation, stimulation of cell migration and neurite retraction, gap junction closure, and even slime mold chemotaxis (Goetzl, et al. (2002), Scientific World Journal, vol. 2: 324-338). The body of knowledge about the biology of LPA continues to grow as more and more cellular systems are tested for LPA responsiveness. For instance, it is now known that, in addition to stimulating cell growth and proliferation, LPA promote cellular tension and cell-surface fibronectin binding, which are important events in wound repair and regeneration (Moolenaar, et al. (2004), BioEssays, vol. 26: 870-881). Recently, anti-apoptotic activity has also been ascribed to LPA, and it has recently been reported that peroxisome proliferation receptor gamma is a receptor/target for LPA (Simon, et al. (2005), J Biol Chem, vol. 280: 14656-14662).

LPA is synthesized by astrocytes, choroid plexus cells and inflammatory cells and is released upon activation; its concentrations increase during inflammation, clotting and trauma. LPA has been clearly identified to have widespread developmental, physiological and pathological actions, controlling events within the nervous, reproductive, gastrointestinal, and vascular systems, and also having a prominent role in cancer, early mammalian embryogenesis and stem cells. In the CNS, for example, LPA levels increase in pathological conditions where the blood brain barrier integrity is damaged, making it a significant factor contributing to the inflammatory response during neurotrauma.

C. Inhibitors of LPA

Inhibitors of LPA are agents that interfere with LPA activity or lower the effective concentration of LPA, typically but not necessarily under physiological conditions. LPA activity may be blocked by direct and/or indirect methods. Indirect methods employ agents that inhibit LPA action on receptors, inhibit LPA biosynthesis or stimulate LPA degradation. Inhibitors of enzymes such as autotaxin (ATX) that are involved in LPA synthesis are examples of indirect inhibitors of LPA. Direct methods of LPA inhibition employ agents that directly bind to and inhibit the activity or effective concentration of LPA. Antibodies to LPA are among the direct inhibitors of LPA. Examples of LPA inhibitors are disclosed in U.S. Pat. Nos. and publications 7,494,775, 7,470,509, 5,994,141, 20100291068, 20100034814, 20100003682, 20090136961, 20080071116, 20050214831, 20040137541, 20030113928, 20030087250, 20020182619, 20020150955, 20020123084, 7,947,665, 7,217,704, 6,875,757, 20100261681, 20090029949, 20080090783, 20070078111, 20060270634, 20060009507, 20050261252, 20040220149, 20040204383, 20030130237, 20030027800, 7,820,703, 7,169,818, 20050107447, 20040122236, 20090197835, 20090062238, 20080318901, 7,459,285, 7,989,663, 20100330143, 20100261681, 20100260682, 20090068697, 20080025950, 20070123492, 20040171096, 20110082181, 20110082164, 20100311799 and 20100152257.

i. Antibodies to LPA

Although polyclonal antibodies against naturally-occurring LPA have been reported in the literature (Chen, et al., Bioorg Med Chem Lett. 2000 Aug. 7; 10(15):1691-3), monoclonal antibodies to LPA had not been described until Sabbadini, et al., U.S. patent application publication no. 20080145360, published Jun. 19, 2008 (attorney docket no. LPT-3100-UT4), and U.S. patent application publication no. 20090136483 (attorney docket no. LPT-3200-UT), published May 28, 2009, both of which are herein incorporated by reference in their entirety for all purposes. The former publication describes the production and characterization of a series of murine monoclonal antibodies against LPA and the latter publication describes a humanized monoclonal antibody against LPA. Additional humanized monoclonal antibodies against LPA are disclosed in U.S. patent application Ser. No. 12/761,584, filed Apr. 16, 2010 (attorney docket no. LPT-3210-UT), the contents of which are also incorporated herein in their entirety. These anti-LPA antibodies are highly specific to LPA. Unlike protein targets, bioactive lipids such as LPA are identical across species. Thus, antibodies that show efficacy and safety in animal models are more easily translated into humans. Antibody drugs are substantially safer than small molecular drugs. Preliminary 7-day toxicology studies performed with murine anti-LPA mAbs show an excellent safety profile commensurate with the fact that the antibodies are highly specific to the bioactive lipid, LPA, and also do not crossreact to any protein epitopes in Western blots of tissue arrays or in IHC.

D. The Role of LPA in Stem Cell Differentiation and in Stem Cell Therapy

i. Neuronal Stem Cells and Therapy

The role of LPA in neuronal differentiation is the best studied of the various systems in which LPA is believed to play a role in differentiation. Neural stem cells (NSC) are found in areas of neurogenesis in the central nervous system (CNS) and can migrate to sites of neural injury. Thus, NSC are under study with the goal of replacing neurons and restoring connections in a neurodegenerative environment. Dottori, et al. (2008), Stem Cells 26(5): 1146-1154.

Following nervous system injury, hemorrhage or trauma, levels of LPA within those tissues increase. Tigyi, et al., Am J Physiol., 1995 May; 268(5 Pt 2):H2048-55; Steiner, et al. (2002), Biochimica et Biophysica Acta (BBA)-Molecular and Cell Biology of Lipids 1582: 154-160. It has been shown that LPA levels equivalent to those reached after injury can inhibit neuronal differentiation of human NSC, suggesting that high levels of LPA within the CNS following injury might inhibit differentiation of NSC into neurons, thus inhibiting endogenous neuronal regeneration. Modulating LPA signaling may thus have a significant impact in nervous system injury, allowing new potential therapeutic approaches.

The present invention concerns enhancing or improving stem cell therapy for nervous system injury by administering an inhibitor of LPA in conjunction or combination with the stem cells, in order to neutralize or decrease levels of LPA in the environment surrounding the stem cells.

LPA is a causal player in the outcome of neural damage and/or repair following injuries. LPA has been identified as a potent inhibitor of neuronal differentiation of neural stem/progenitor cells (NS/PC), effects very likely to be relevant to several neuro-pathophysiologies, including TBI. LPA is hypothesized to have two mechanisms of action that contribute to poor outcomes: (1) LPA is a pro-inflammatory and thrombogenic mediator that participates in the early responses to injury eventually resulting in neural cell necrosis and gliosis; and (2) LPA inhibits neural tissue regenerative responses by interfering with NS/PC activity. An upregulation of LPA receptor 2 (LPAR₂) has been shown following injury in the adult mouse CNS and human brain. Frugier, et al. (2011) Cell Mol. Neurobiol. 31:569-577. Thus, LPA dysregulation/upregulation may contribute to the progression of the injury.

It has been suggested that LPA is one of the key signaling molecules that is upregulated during injury, neurodegeneration, and ischemia to actively inhibit stem cell activity is the bioactive lipid. In the CNS, LPA levels increase in pathological conditions where the blood brain barrier integrity is damaged, making it a significant factor contributing to an inflammatory response, gliosis, and neuronal death during neurotrauma. The role of LPA in this context has been elucidated using a model system of neuronal differentiation. See Dottori, et al. This work demonstrates that LPA inhibits NS/PC activity and that LPA levels are elevated following injury. Thus, it is believed that LPA release at injury sites inhibits the neuronal differentiation of NS/PC.

Following spinal cord injury, levels of the inflammatory lipid, LPA, increase in the central nervous system (CNS) and at the site of injury. LPA strongly contributes to the non-regeneration of neurons, increases inflammation, and inhibits neural stem cell differentiation into neurons. These effects are highly deleterious to the spinal cord.

Traumatic brain injury (TBI) is a disruption of function in the brain that results from a blow or jolt to the head or penetrating head injury. There are more than 1.5 million TBIs per year in the US, with 125,000 of these resulting in permanent disability. Moreover, TBI is the leading cause of military casualties in the field (150,000 from Iraq and Afghanistan to date) and a leading source of long-term rehabilitation problems suffered by veterans. When not fatal (22% of moderate and 35% of severe TBI patients die within the first year following injury), TBI can result in permanent and severe physical, cognitive, and behavioural impairments, leaving sufferers in need of long term healthcare. Currently, there are no FDA-approved drugs targeting TBI. It is believed that interfering directly or indirectly with LPA as part of stem cell therapy will dramatically enhance repair, e.g. following traumatic injury (TBI) or spinal cord injury (SCI).

Neuronal stem cells have the option of proceeding into neuronal differentiation or into glial differentiation (gliogenesis), the formation of non-neuronal glial cells. Macroglial cells (glia) include astrocytes and oligodendrocytes. Thus, in general, as neuronal differentiation increases, glial differentiation decreases and vice versa. Thus, an increase in neuronal differentiation may be determined by an increase in neuron formation, or by a decrease in glial differentiation. NSC can be maintained in vitro as floating neurospheres and can differentiate in vitro into neurons. This can be assayed by visualizing and quantitating neuronal outgrowth from the neurospheres, which is visible under a microscope. This provides an elegant system for observing the effect of anti-LPA antibodies or other LPA inhibitors on neuronal differentiation.

Stem cell therapy incorporating anti-LPA inhibition will also be efficacious in numerous neurodegenerative conditions, such as Alzheimer's disease, Parkinson's disease, and others. Among such embodiments are combination therapies that can be useful in a broad spectrum of neurodegenerative diseases.

ii. Cardiac Stem Cells and Therapies

a. Myocardial regeneration after infarction:Ischemic myocardial infarction results in loss of myocardial tissue and commensurate negative remodelling of tissue and loss in heart function. Thus, use of stem cell transplantation to regenerate cardiac tissue post-myocardial infarction is a topic of intense investigation (Shah and Shalia, Stem Cells Int. 2011; 2011:536758. Epub 2011 May 11). As is the case for many other contemplated uses of stem cell transplants to regenerate tissue, there are many challenges to successful transplantation including methods to ensure successful retention and survival of the exogenously transplanted cells in cardiac tissue. The source of stem cells for even allogeneic transplantation is challenging for most tissues such as cardiac, skeletal muscle and brain as it is difficult to harvest stem cells from those tissues for cell culture and subsequent allograft. Stem cells derived from easily accessible tissues such as bone marrow and adipose tissue may be used. In addition, the stem cell precursors or stem cells derived from non-cardiac sources such as bone marrow (BMCs) or adipose tissue (ASCs) must differentiate into the tissue of choice once transplanted and seeded. Adipose tissue-derived stem cells have been used for autologous stem cell transplantation and illustrate the capability for transdifferentiation of these stem cells into cardiomyocytes. However, survival of these transplants has been poor. Co-administration of biopolymers has been used to improve the retention of adipose-derived stem cells in rats post-MI (Danoviz et al PLoS One. 2010 Aug. 10; 5(8):e12077). In the context of the invention is believed that inhibition of LPA may enhance the survival and effectiveness of stem cell therapy for treatment of cardiac disease including myocardial infarction.

b. Myocardial regeneration after cancer chemotherapy or in end-stage heart failure:Stem cell transplantation is envisioned in the treatment of heart failure due to not only due to MI but also of end-stage heart failure of any cause (e.g. viral myocarditis, chemotherapy, idiopathic heart failure). For example, cancer chemotherapeutic agents such as doxorubicin, camptothecin, and thapsigargin have well-known side effects on the heart that result in heart failure due to the death of cardiomyocytes (Maney, et al. Cardiovasc Toxicol. 2011 September; 11(3):253-62). Thus, stem cell therapy is envisioned to prevent negative cardiac remodelling associated with chemotherapy-induced cell death in the heart. Because of the beneficial effects of neutralizing LPA on stem cells, it is believed that inhibition of LPA will enhance the effectiveness of stem cell therapy for heart failure.

c. Stem cell-directed angiogenesis as a treatment for the ischemic heart:Stem cells have been used to induce therapeutic angiogenesis after myocardial ischemia. Mesenchymal stem cells transplanted in rats after cardial ischemia resulted in enhanced heart function and a small number of the stem cells differentiated into cardiomyocytes. Capillary density was also higher in the stem cell transplanted hearts than in controls. Tang, et al. (2006) Eur. J. Cardio-thoracic Surg. 30:353-361. In the context of the invention, endothelial precursor cells (EPCs), for example, can also be used to promote angiogenesis in the heart, e.g., after MI or non-MI acute coronary syndrome (ACS).

Endothelial precursor cells (EPCs) may be used to promote angiogenesis in the heart after MI or non-MI acute coronary syndrome (ACS). EPCs could be isolated and cultured in vitro and then allografted into a large experimental animal such as pigs by cardiac catheterization. Animals could be treated with anti-LPA mAbs by systemic administration as well as co-administration with the EPCs with the catheter. Seeded EPCs would then promote neovascularization to improve blood flow after an ischemic event. Ischemia can be induced either by surgical coronary ligation, thermocoagulation using a cardiac catheter or by use of an ameroid ring surgically placed around a coronary vessel to promote constriction and subsequent ischemia.

iii. Stem Cell Therapy for Neuromuscular Disorders

The muscular dystrophies, such as Duchenne muscular dystrophy (DMD) are candidates for stem cell therapy (reviewed by Negroni, et al., Expert Opin Biol Ther. 2011 February; 11(2):157-76). There are no effective medical treatments for DMD and related genetic disorders. A variety of stem cell sources have been proposed, including myoblasts, mesoangioblasts, pericytes, myoendothelial cells, CD133+ cells, aldehyde dehydrogenase-positive cells, mesenchymal stem cells, embryonic stem cells and induced pluripotent stem cells usually for direct injection into muscle tissue. The mdx strain of mice is commonly used as a model of DMD. Several clinical trials have been attempted with little success in recovering muscle function and in restoring the expression of the missing dysrophin protein (reviewed by Negroni et al., ibid). It is believed that co-administration of anti-LPA inhibitor either at the point of cell injection or systemically, or both, could improve the regenerative capacity of implanted cells.

A phase I trial of spinal cord derived stem cells for patients with ALS has recently begun at Emory University, sponsored by Neuralstem, Inc. While the efforts in stem cell treatment for ALS are very preliminary, stem cell therapy for this condition that incorporates inhibition of LPA will be therapeutically useful.

iv. Stem Cell Therapy for Treatment of Diabetes and Related Disorders:

Type I diabetics suffer from insulin deficiency due to the loss of pancreatic beta cells of the islets of Langerhans. Allogeneic islet cell transplantation for the treatment of type 1 diabetes, and autologous islet cell transplantation for the prevention of surgical diabetes after a total pancreatectomy (such as in treatment of pancreatitis) are being attempted and these approaches can potentially be enhanced by addition of anti-LPA inhibition. The challenges and successes of stem cell therapy for Type 1 diabetics has recently been reviewed (Aguaye-Mazzucato, et al., Nat Rev Endocrinol. 2010 March; 6(3):139-48). As is commonly the case for stem cell therapy, conversion of precursor cells into glucose-induced insulin-producing islet cells has not yet been perfected and new strategies are needed. One such new strategy proposed herein is the co-administration of anti-LPA inhibition to enhance stem cell efficacy, e.g., by promotion of differentiation, such as transdifferentiation. The preferred animal model for Type 1 diabetes is the STZ rat in which streptozotocin treatment results in the death of islet beta cells and the development of glucose intolerance. Islet precursor cells have been used in this model to restore islet cell function (Li, et al., Acta Pharmacol Sin. 2010 November; 31(11):1454-63. Epub 2010 Oct. 18). Additionally, intracavernous transplantation of bone marrow-derived mesenchymal stem cells has been used to restore erectile function of streptotozocin-induced diabetic rats. (Jin, et al., Transplant Proc. 2010 September; 42(7):2745-52).

3. Definitions

Before describing the instant invention in detail, several terms used in the context of the present invention will be defined. In addition to these terms, others are defined elsewhere in the specification, as necessary. Unless otherwise expressly defined herein, terms of art used in this specification will have their art-recognized meanings.

An “allograft” or “allogeneic transplant” is a transplantation of cells, tissues or organs from a genetically non-identical member of the same species as the recipient.

The term “antibody” (“Ab”) or “immunoglobulin” (Ig) refers to any form of a peptide, polypeptide derived from, modeled after or encoded by, an immunoglobulin gene, or fragment thereof, that is capable of binding an antigen or epitope. See, e.g., Immunobiology, Fifth Edition, C. A. Janeway, P. Travers, M., Walport, M. J. Shlomchiked., ed. Garland Publishing (2001).

An “antibody derivative” is an immune-derived moiety, i.e., a molecule that is derived from an antibody. This comprehends, for example, antibody variants, antibody fragments, chimeric antibodies, humanized antibodies, multivalent antibodies, antibody conjugates and the like, which retain a desired level of binding activity for antigen.

As used herein, “antibody fragment” refers to a portion of an intact antibody that includes the antigen binding site or variable regions of an intact antibody, wherein the portion can be free of the constant heavy chain domains (e.g., CH2, CH3, and CH4) of the Fc region of the intact antibody. Alternatively, portions of the constant heavy chain domains (e.g., CH2, CH3, and CH4) can be included in the “antibody fragment”. Antibody fragments retain antigen binding ability and include Fab, Fab”, F(ab′)₂, Fd, and Fv fragments; diabodies; triabodies; single-chain antibody molecules (sc-Fv); minibodies, nanobodies, and multispecific antibodies formed from antibody fragments. Papain digestion of antibodies produces two identical antigen-binding fragments, called “Fab” fragments, each with a single antigen-binding site, and a residual “Fc” fragment, whose name reflects its ability to crystallize readily. Pepsin treatment yields an F(ab′)₂ fragment that has two antigen-combining sites and is still capable of cross-linking antigen. By way of example, a Fab fragment also contains the constant domain of a light chain and the first constant domain (CH1) of a heavy chain. “Fv” is the minimum antibody fragment that contains a complete antigen-recognition and -binding site. This region consists of a dimer of one heavy chain and one light chain variable domain in tight, non-covalent association. It is in this configuration that the three hypervariable regions of each variable domain interact to define an antigen-binding site on the surface of the V_{H}-V_{L} dimer. Collectively, the six hypervariable regions confer antigen-binding specificity to the antibody. However, even a single variable domain (or half of an Fv comprising only three hypervariable regions specific for an antigen) has the ability to recognize and bind antigen, although at a lower affinity than the entire binding site. “Single-chain Fv” or “sFv” antibody fragments comprise the V_{H} and V_{L} domains of antibody, wherein these domains are present in a single polypeptide chain. Generally, the Fv polypeptide further comprises a polypeptide linker between the V_{H} and V_{L} domains that enables the sFv to form the desired structure for antigen binding. For a review of sFv, see Pluckthun in The Pharmacology of Monoclonal Antibodies, vol. 113, Rosenburg and Moore eds. Springer-Verlag, New York, pp. 269-315 (1994).

The Fab fragment also contains the constant domain of the light chain and the first constant domain (CH1) of the heavy chain. Fab′ fragments differ from Fab fragments by the addition of a few residues at the carboxyl terminus of the heavy chain CH1 domain including one or more cysteine(s) from the antibody hinge region. Fab′-SH is the designation herein for Fab′ in which the cysteine residue(s) of the constant domains bear a free thiol group. F(ab′)₂ antibody fragments originally were produced as pairs of Fab′ fragments which have hinge cysteines between them. Other chemical couplings of antibody fragments are also known.

An “antibody variant” refers herein to a molecule which differs in amino acid sequence from a native antibody (e.g., an anti-LPA antibody) amino acid sequence by virtue of addition, deletion and/or substitution of one or more amino acid residue(s) in the antibody sequence and which retains at least one desired activity of the parent anti-binding antibody. Desired activities can include the ability to bind the antigen specifically, the ability to inhibit proliferation in vitro, the ability to inhibit angiogenesis in vivo, and the ability to alter cytokine profile in vitro. The amino acid change(s) in an antibody variant may be within a variable region or a constant region of a light chain and/or a heavy chain, including in the Fc region, the Fab region, the CH₁ domain, the CH₂ domain, the CH₃ domain, and the hinge region. In one embodiment, the variant comprises one or more amino acid substitution(s) in one or more hypervariable region(s) of the parent antibody. For example, the variant may comprise at least one, e.g. from about one to about ten, and preferably from about two to about five, substitutions in one or more hypervariable regions of the parent antibody. Ordinarily, the variant will have an amino acid sequence having at least 75% amino acid sequence identity with the parent antibody heavy or light chain variable domain sequences, more preferably at least 65%, more preferably at 80%, more preferably at least 85%, more preferably at least 90%, and most preferably at least 95%. Identity or homology with respect to this sequence is defined herein as the percentage of amino acid residues in the candidate sequence that are identical with the parent antibody residues, after aligning the sequences and introducing gaps, if necessary, to achieve the maximum percent sequence identity. None of N-terminal, C-terminal, or internal extensions, deletions, or insertions into the antibody sequence shall be construed as affecting sequence identity or homology. The variant retains the ability to bind LPA and preferably has desired activities which are superior to those of the parent antibody. For example, the variant may have a stronger binding affinity, enhanced ability to reduce angiogenesis and/or halt tumor progression. To analyze such desired properties (for example les immunogenic, longer half-life, enhanced stability, enhanced potency), one should compare a Fab form of the variant to a Fab form of the parent antibody or a full length form of the variant to a full length form of the parent antibody, for example, since it has been found that the format of the anti-sphingolipid antibody impacts its activity in the biological activity assays disclosed herein. The variant antibody of particular interest herein can be one which displays at least about 10 fold, preferably at least about % 5, 25, 59, or more of at least one desired activity. The preferred variant is one that has superior biophysical properties as measured in vitro or superior activities biological as measured in vitro or in vivo when compared to the parent antibody.

The term “antigen” refers to a molecule that is recognized and bound by an antibody molecule or immune-derived moiety that binds to the antigen. The specific portion of an antigen that is bound by an antibody is termed the “epitope.”

An “anti-LPA antibody” refers to any antibody or antibody-derived molecule that binds lysophosphatidic acid. The terms “anti-LPA antibody,” “antibody that binds LPA” and “antibody reactive with LPA” are interchangeable.

An “autograft” or “autologous transplant” refers to transplantation of a subject's own cells or tissues (e.g., bone marrow) back into the subject, generally after some kind of treatment.

A “bioactive lipid” refers to a lipid signaling molecule. Bioactive lipids are distinguished from structural lipids (e.g., membrane-bound phospholipids) in that they mediate extracellular and/or intracellular signaling and thus are involved in controlling the function of many types of cells by modulating differentiation, migration, proliferation, secretion, survival, and other processes.

The term “biologically active,” in the context of an antibody or antibody fragment or variant, refers to an antibody or antibody fragment or antibody variant that is capable of binding the desired epitope and in some ways exerting a biologic effect. Biological effects include, but are not limited to, the modulation of a growth signal, the modulation of an anti-apoptotic signal, the modulation of an apoptotic signal, the modulation of the effector function cascade, and modulation of other ligand interactions.

A “biomarker” is a specific biochemical in the body which has a particular molecular feature that makes it useful for measuring the progress of disease or the effects of treatment.

A “carrier” refers to a moiety adapted for conjugation to a hapten, thereby rendering the hapten immunogenic. A representative, non-limiting class of carriers is proteins, examples of which include albumin, keyhole limpet hemocyanin, hemaglutanin, tetanus, and diptheria toxoid. Other classes and examples of carriers suitable for use in accordance with the invention are known in the art. These, as well as later discovered or invented naturally occurring or synthetic carriers, can be adapted for application in accordance with the invention.

The term “chemotherapeutic agent” means anti-cancer and other anti-hyperproliferative agents. Thus chemotherapeutic agents are a subset of therapeutic agents in general. Chemotherapeutic agents include, but are not limited to: DNA damaging agents and agents that inhibit DNA synthesis: anthracyclines (doxorubicin, donorubicin, epirubicin), alkylating agents (bendamustine, busulfan, carboplatin, carmustine, chlorambucil, cyclophosphamide, dacarbazine, hexamethylmelamine, ifosphamide, lomustine, mechlorethamine, melphalan, mitotane, mytomycin, pipobroman, procarbazine, streptozocin, thiotepa, and triethylenemelamine), platinum derivatives (cisplatin, carboplatin, cis diammine-dichloroplatinum), and topoisomerase inhibitors (Camptosar); anti-metabolites such as capecitabine, chlorodeoxyadenosine, cytarabine (and its activated form, ara-CMP), cytosine arabinoside, dacabazine, floxuridine, fludarabine, 5-fluorouracil, 5-DFUR, gemcitabine, hydroxyurea, 6-mercaptopurine, methotrexate, pentostatin, trimetrexate, 6-thioguanine); anti-angiogenics (bevacizumab, thalidomide, sunitinib, lenalidomide, TNP-470, 2-methoxyestradiol, ranibizumab, sorafenib, erlotinib, bortezomib, pegaptanib, endostatin); vascular disrupting agents (flavonoids/flavones, DMXAA, combretastatin derivatives such as CA4DP, ZD6126, AVE8062A, etc.); biologics such as antibodies (Herceptin, Avastin, Panorex, Rituxin, Zevalin, Mylotarg, Campath, Bexxar, Erbitux); endocrine therapy: aromatase inhibitors (4-hydroandrostendione, exemestane, aminoglutehimide, anastrazole, letozole), anti-estrogens (Tamoxifen, Toremifine, Raoxifene, Faslodex), steroids such as dexamethasone; immuno-modulators: cytokines such as IFN-beta and IL2), inhibitors to integrins, other adhesion proteins and matrix metalloproteinases); histone deacetylase inhibitors like suberoylanilide hydroxamic acid; inhibitors of signal transduction such as inhibitors of tyrosine kinases like imatinib (Gleevec); inhibitors of heat shock proteins like 17-N-allylamino-17-demethoxygeldanamycin; retinoids such as all trans retinoic acid; inhibitors of growth factor receptors or the growth factors themselves; anti-mitotic compounds and/or tubulin-depolymerizing agents such as the taxoids (paclitaxel, docetaxel, taxotere, BAY 59-8862), navelbine, vinblastine, vincristine, vindesine and vinorelbine; anti-inflammatories such as COX inhibitors and cell cycle regulators, e.g., check point regulators and telomerase inhibitors.

The term “chimeric” antibody (or immunoglobulin) refers to a molecule comprising a heavy and/or light chain which is identical with or homologous to corresponding sequences in antibodies derived from a particular species or belonging to a particular antibody class or subclass, while the remainder of the chain(s) is identical with or homologous to corresponding sequences in antibodies derived from another species or belonging to another antibody class or subclass, as well as fragments of such antibodies, so long as they exhibit the desired biological activity (Cabilly, et al., infra; Morrison et al., Proc. Natl. Acad. Sci. U.S.A., vol. 81:6851 (1984)).

The term “combination therapy” refers to a therapeutic regimen that involves the provision of at least two distinct therapies to achieve an indicated therapeutic effect. For example, a combination therapy may involve the administration of two or more chemically distinct active ingredients, for example, a stem cell composition and an agent that inhibits LPA (e.g., and anti-LPA mAb or an LPA-binding antigen binding fragment derived from such an antibody). Alternatively, a combination therapy may involve the administration of an anti-LPA agent and a stem cell composition in conjunction with the delivery of another treatment, such as radiation therapy and/or surgery. In particularly preferred embodiments, combination therapy may involve the therapeutic administration of stem cells and an antibody (or antigenbinding fragment thereof) that binds and neutralizes LPA. In the context of the administration of a combination therapy, it is understood that the active ingredients (i.e., a stem cell composition and an anti-LPA agent) may be administered as part of the same composition or as different compositions. When administered as separate compositions, the compositions comprising the different active ingredients may be administered at the same or different times, in the same or different number of doses, by the same or different routes, using the same of different dosing regimens, all as the particular context requires and as determined by the attending physician. Similarly, when one or more anti-LPA agents, for example, an anti-LPA antibody, is used in combination with stem cells, and in some cases, also radiation and/or surgery, the anti-LPA species may be delivered before, at the same time, or after the stem cells, surgery, and/or or radiation treatment.

The term “diabodies” refers to small antibody fragments with two antigen-binding sites, which fragments comprise a heavy chain variable domain (V_{H}) connected to a light chain variable domain (V_{L}) in the same polypeptide chain (V_{H}-V_{L}). By using a linker that is too short to allow pairing between the two domains on the same chain, the domains are forced to pair with the complementary domains of another chain and create two antigen-binding sites. Diabodies are described more fully in, for example, EP 404,097; WO 93/11161; and Hollinger, et al., Proc. Natl. Acad. Sci. USA 90:6444-6448 (1993).

“Effective concentration” refers to the absolute, relative, and/or available concentration and/or activity, for example of certain undesired bioactive lipids. In other words, the effective concentration of a bioactive lipid is the amount of lipid available, and able, to perform its biological function. For example, a monoclonal antibody directed to a bioactive lipid (such as, for example, LPA) is able to reduce the effective concentration of the lipid by binding to the lipid and rendering it unable to perform its biological function. In this example, the lipid itself is still present (it is not degraded by the antibody, in other words) but can no longer bind its receptor or other targets to cause a downstream effect, so “effective concentration” rather than absolute concentration is the appropriate measurement. Lowering the effective concentration of the bioactive lipid may be said to “neutralize” the target lipid. Methods and assays exist for directly and/or indirectly measuring the effective concentration of bioactive lipids.

An “epitope” or “antigenic determinant” refers to that portion of an antigen that reacts with an antibody antigen-binding portion derived from an antibody.

A “fully human antibody” can refer to an antibody produced in a genetically engineered (i.e., transgenic) mouse (e.g., from Medarex) that, when presented with an immunogen, can produce a human antibody that does not necessarily require CDR grafting. These antibodies are fully human (100% human protein sequences) from animals such as mice in which the non-human antibody genes are suppressed and replaced with human antibody gene expression. The applicants believe that antibodies could be generated against bioactive lipids when presented to these genetically engineered mice or other animals that might be able to produce human frameworks for the relevant CDRs.

A “hapten” is a substance that is non-immunogenic but can react with an antibody or antigen-binding portion derived from an antibody. In other words, haptens have the property of antigenicity but not immunogenicity. A hapten is generally a small molecule that can, under most circumstances, elicit an immune response (i.e., act as an antigen) only when attached to a carrier, for example, a protein, polyethylene glycol (PEG), colloidal gold, silicone beads, or the like. The carrier may be one that also does not elicit an immune response by itself.

The term “heteroconjugate antibody” can refer to two covalently joined antibodies. Such antibodies can be prepared using known methods in synthetic protein chemistry, including using crosslinking agents. As used herein, the term “conjugate” refers to molecules formed by the covalent attachment of one or more antibody fragment(s) or binding moieties to one or more polymer molecule(s).

“Humanized” forms of non-human (e.g., murine) antibodies are chimeric antibodies that contain minimal sequence derived from non-human immunoglobulin. Or, looked at another way, a humanized antibody is a human antibody that also contains selected sequences from non-human (e.g., murine) antibodies in place of the human sequences. A humanized antibody can include conservative amino acid substitutions or non-natural residues from the same or different species that do not significantly alter its binding and/or biologic activity. Such antibodies are chimeric antibodies that contain minimal sequence derived from non-human immunoglobulins. For the most part, humanized antibodies are human immunoglobulins (recipient antibody) in which residues from a complementary-determining region (CDR) of the recipient are replaced by residues from a CDR of a non-human species (donor antibody) such as mouse, rat, camel, bovine, goat, or rabbit having the desired properties. In some instances, framework region (FR) residues of the human immunoglobulin are replaced by corresponding non-human residues. The CDRs can be placed into any of a variety of frameworks as long as a desired level of antigen binding is retained.

Furthermore, humanized antibodies can comprise residues that are found neither in the recipient antibody nor in the imported CDR or framework sequences. These modifications are made to further refine and maximize antibody performance. Thus, in general, a humanized antibody will comprise all of at least one, and in one aspect two, variable domains, in which all or all of the hypervariable loops correspond to those of a non-human immunoglobulin and all or substantially all of the FR regions are those of a human immunoglobulin sequence. The humanized antibody optionally also will comprise at least a portion of an immunoglobulin constant region (Fc), or that of a human immunoglobulin. See, e.g., Cabilly, et al., U.S. Pat. No. 4,816,567; Cabilly, et al., European Patent No. 0,125,023 B1; Boss, et al., U.S. Pat. No. 4,816,397; Boss, et al., European Patent No. 0,120,694 B1; Neuberger, et al., WO 86/01533; Neuberger, et al., European Patent No. 0,194,276 B1; Winter, U.S. Pat. No. 5,225,539; Winter, European Patent No. 0,239,400 B1; Padlan, et al., European Patent Application No. 0,519,596 A1; Queen, et al. (1989), Proc. Nat'l Acad. Sci. USA, vol. 86:10029-10033). For further details, see Jones et al., Nature 321:522-525 (1986); Reichmann et al., Nature 332:323-329 (1988); and Presta, Curr. Op. Struct. Biol. 2:593-596 (1992) and Hansen, WO2006105062.

The term “hyperproliferative disorder” refers to diseases and disorders associated with, the uncontrolled proliferation of cells, including but not limited to uncontrolled growth of organ and tissue cells resulting in cancers and benign tumors. Hyperproliferative disorders associated with endothelial cells can result in diseases of angiogenesis such as angiomas, endometriosis, obesity, age-related macular degeneration and various retinopathies, as well as the proliferation of endothelial cells and smooth muscle cells that cause restenosis as a consequence of stenting in the treatment of atherosclerosis. Hyperproliferative disorders involving fibroblasts (i.e., fibrogenesis) include but are not limited to disorders of excessive scarring (i.e., fibrosis) such as age-related macular degeneration, cardiac remodeling and failure associated with myocardial infarction, excessive wound healing such as commonly occurs as a consequence of surgery or injury, keloids, and fibroid tumors and stenting.

An “immunogen” is a molecule capable of inducing a specific immune response, particularly an antibody response in an animal to whom the immunogen has been administered. In the instant invention, the immunogen is a derivatized bioactive lipid conjugated to a carrier, i.e., a “derivatized bioactive lipid conjugate”. The derivatized bioactive lipid conjugate used as the immunogen may be used as capture material for detection of the antibody generated in response to the immunogen. Thus the immunogen may also be used as a detection reagent. Alternatively, the derivatized bioactive lipid conjugate used as capture material may have a different linker and/or carrier moiety from that in the immunogen.

To “inhibit,” particularly in the context of a biological phenomenon, means to decrease, suppress or delay. For example, a treatment yielding “inhibition of tumorigenesis” may mean that tumors do not form at all, or that they form more slowly, or are fewer in number than in the untreated control.

An “inhibitor of LPA” or “LPA inhibitor” is an agent that interferes with LPA activity and/or lowers the effective concentration of LPA, typically but not necessarily under physiological conditions. Similarly, “inhibition of LPA” or “LPA inhibition” refers to interference with LPA activity or reduction in the effective concentration of LPA. LPA inhibition may be achieved by direct and/or indirect methods. “Indirect inhibition” of LPA employs agents that inhibit LPA action on receptors, inhibit LPA biosynthesis or stimulate LPA degradation. Inhibitors of enzymes such as autotaxin (ATX) that are involved in LPA synthesis are examples of indirect inhibitors of LPA. “Direct inhibition” of LPA inhibition employs agents that directly bind to and inhibit the activity or effective concentration of LPA. Antibodies to LPA are among the direct inhibitors of LPA.

An “isolated” antibody is one that has been identified and separated and/or recovered from a component of its natural environment. Contaminant components of its natural environment are materials that would interfere with diagnostic or therapeutic uses for the antibody, and may include enzymes, hormones, and other proteinaceous or nonproteinaceous solutes. In preferred embodiments, the antibody will be purified (1) to greater than 95% by weight of antibody as determined by the Lowry method, and most preferably more than 99% by weight, (2) to a degree sufficient to obtain at least 15 residues of N-terminal or internal amino acid sequence by use of a spinning cup sequenator, or (3) to homogeneity by SDS-PAGE under reducing or nonreducing conditions using Coomassie blue or, preferably, silver stain. Isolated antibody includes the antibody in situ within recombinant cells since at least one component of the antibody's natural environment will not be present. Ordinarily, however, isolated antibody will be prepared by at least one purification step.

The word “label” when used herein refers to a detectable compound or composition, such as one that is conjugated directly or indirectly to the antibody. The label may itself be detectable by itself (e.g., radioisotope labels or fluorescent labels) or, in the case of an enzymatic label, may catalyze chemical alteration of a substrate compound or composition that is detectable.

The expression “linear antibodies” when used throughout this application refers to the antibodies described in Zapata et al. Protein Eng. 8(10):1057-1062 (1995). Briefly, these antibodies comprise a pair of tandem Fd segments (V_{H}-C_{H}1-V_{H}-C_{H}1) that form a pair of antigen binding regions. Linear antibodies can be bispecific or monospecific.

In the context of this invention, a “liquid composition” refers to one that, in its filled and finished form as provided from a manufacturer to an end user (e.g., a doctor or nurse), is a liquid or solution, as opposed to a solid. Here, “solid” refers to compositions that are not liquids or solutions. For example, solids include dried compositions prepared by lyophilization, freeze-drying, precipitation, and similar procedures.

The term “metabolites” refers to compounds from which LPAs are made, as well as those that result from the degradation of LPAs; that is, compounds that are involved in the lysophospholipid metabolic pathways. The term “metabolic precursors” may be used to refer to compounds from which sphingolipids are made.

The term “monoclonal antibody” (mAb) as used herein refers to an antibody obtained from a population of substantially homogeneous antibodies, or to said population of antibodies. The individual antibodies comprising the population are essentially identical, except for possible naturally occurring mutations that may be present in minor amounts. Monoclonal antibodies are highly specific, being directed against a single antigenic site. Furthermore, in contrast to conventional (polyclonal) antibody preparations that typically include different antibodies directed against different determinants (epitopes), each monoclonal antibody is directed against a single determinant on the antigen. The modifier “monoclonal” indicates the character of the antibody as being obtained from a substantially homogeneous population of antibodies, and is not to be construed as requiring production of the antibody by any particular method. For example, monoclonal antibodies to be used in accordance with the present invention may be made by the hybridoma method first described by Kohler, et al., Nature 256:495 (1975), or may be made by recombinant DNA methods (see, e.g., U.S. Pat. No. 4,816,567). “Monoclonal antibodies” may also be isolated from phage antibody libraries using the techniques described in Clackson, et al., Nature 352:624-628 (1991) and Marks, et al., J. Mol. Biol. 222:581-597 (1991), for example, or by other methods known in the art. The monoclonal antibodies herein specifically include chimeric antibodies in which a portion of the heavy and/or light chain is identical with or homologous to corresponding sequences in antibodies derived from a particular species or belonging to a particular antibody class or subclass, while the remainder of the chain(s) is identical with or homologous to corresponding sequences in antibodies derived from another species or belonging to another antibody class or subclass, as well as fragments of such antibodies, so long as they exhibit the desired biological activity (U.S. Pat. No. 4,816,567; and Morrison, et al., Proc. Natl. Acad. Sci. USA 81:6851-6855 (1984)).

“Monotherapy” refers to a treatment regimen based on the delivery of one therapeutically effective compound, whether administered as a single dose or several doses over time.

The term “multispecific antibody” can refer to an antibody, or a monoclonal antibody, having binding properties for at least two different epitopes. In one embodiment, the epitopes are from the same antigen. In another embodiment, the epitopes are from two or more different antigens. Methods for making multispecific antibodies are known in the art. Multispecific antibodies include bispecific antibodies (having binding properties for two epitopes), trispecific antibodies (three epitopes) and so on. For example, multispecific antibodies can be produced recombinantly using the co-expression of two or more immunoglobulin heavy chain/light chain pairs. Alternatively, multispecific antibodies can be prepared using chemical linkage. One of skill can produce multispecific antibodies using these or other methods as may be known in the art. Multispecific antibodies include multispecific antibody fragments.

“Neoplasia” or “cancer” refers to abnormal and uncontrolled cell growth. A “neoplasm”, or tumor or cancer, is an abnormal, unregulated, and disorganized proliferation of cell growth, and is generally referred to as cancer. A neoplasm may be benign or malignant. A neoplasm is malignant, or cancerous, if it has properties of destructive growth, invasiveness, and metastasis. Invasiveness refers to the local spread of a neoplasm by infiltration or destruction of surrounding tissue, typically breaking through the basal laminas that define the boundaries of the tissues, thereby often entering the body's circulatory system. Metastasis typically refers to the dissemination of tumor cells by lymphatics or blood vessels. Metastasis also refers to the migration of tumor cells by direct extension through serous cavities, or subarachnoid or other spaces. Through the process of metastasis, tumor cell migration to other areas of the body establishes neoplasms in areas away from the site of initial appearance.

“Neural” means pertaining to nerves. Nerves are bundles of fibers made up of neurons.

“Neural stem cells” (NSCs) are the self-renewing, multipotent cells that differentiate into the main phenotypes of the nervous system. NSCs give rise to glial and neuronal cells. Neuronal stem cells give rise to neuronal cells. Neural progenitor cells (NPCs) are the progeny of stem cell division that normally undergo a limited number of replication cycles in vivo.

“Neuron” refers to an excitable cell type in the nervous system that processes and transmits information by electrochemical signalling. Neurons are the core components of the CNS (brain and spinal cord) and the peripheral nerves. “Neuronal” means “pertaining to neurons.”

“Neuronal differentiation” is the conversion of neural stem cells toward the mature cell types of the nervous system, such as neurons, astrocytes, etc. Such differentiation occurs in vivo but can be caused to occur in vitro in model systems such as neurospheres. Differentiation may be a multistep or multistage process and thus multiple phases or steps of differentiation can be studied in vitro.

The “parent” antibody herein is one that is encoded by an amino acid sequence used for the preparation of the variant. The parent antibody may be a native antibody or may already be a variant, e.g., a chimeric antibody. For example, the parent antibody may be a humanized or human antibody.

A “patentable” composition, process, machine, or article of manufacture according to the invention means that the subject matter satisfies all statutory requirements for patentability at the time the analysis is performed. For example, with regard to novelty, non-obviousness, or the like, if later investigation reveals that one or more claims encompass one or more embodiments that would negate novelty, non-obviousness, etc., the claim(s), being limited by definition to “patentable” embodiments, specifically exclude the non-patentable embodiment(s). Also, the claims appended hereto are to be interpreted both to provide the broadest reasonable scope, as well as to preserve their validity. Furthermore, the claims are to be interpreted in a way that (1) preserves their validity and (2) provides the broadest reasonable interpretation under the circumstances, if one or more of the statutory requirements for patentability are amended or if the standards change for assessing whether a particular statutory requirement for patentability is satisfied from the time this application is filed or issues as a patent to a time the validity of one or more of the appended claims is questioned.

The term “pharmaceutically acceptable salt” refers to a salt, such as used in formulation, which retains the biological effectiveness and properties of the agents and compounds of this invention and which are is biologically or otherwise undesirable. In many cases, the agents and compounds of this invention are capable of forming acid and/or base salts by virtue of the presence of charged groups, for example, charged amino and/or carboxyl groups or groups similar thereto. Pharmaceutically acceptable acid addition salts may be prepared from inorganic and organic acids, while pharmaceutically acceptable base addition salts can be prepared from inorganic and organic bases. For a review of pharmaceutically acceptable salts (see Berge, et al. (1977) J. Pharm. Sci., vol. 66, 1-19).

A “plurality” means more than one.

The terms “separated”, “purified”, “isolated”, and the like mean that one or more components of a sample contained in a sample-holding vessel are or have been physically removed from, or diluted in the presence of, one or more other sample components present in the vessel. Sample components that may be removed or diluted during a separating or purifying step include, chemical reaction products, non-reacted chemicals, proteins, carbohydrates, lipids, and unbound molecules.

By “solid phase” is meant a non-aqueous matrix such as one to which the antibody of the present invention can adhere. Examples of solid phases encompassed herein include those formed partially or entirely of glass (e.g. controlled pore glass), polysaccharides (e.g., agarose), polyacrylamides, polystyrene, polyvinyl alcohol and silicones. In certain embodiments, depending on the context, the solid phase can comprise the well of an assay plate; in others it is a purification column (e.g. an affinity chromatography column). This term also includes a discontinuous solid phase of discrete particles, such as those described in U.S. Pat. No. 4,275,149.

The term “species” is used herein in various contexts, e.g., a particular species of chemotherapeutic agent. In each context, the term refers to a population of chemically indistinct molecules of the sort referred in the particular context.

The term “specific” or “specificity” in the context of antibody-antigen interactions refers to the selective, non-random interaction between an antibody and its target epitope. Here, the term “antigen” refers to a molecule that is recognized and bound by an antibody molecule or other immune-derived moiety. The specific portion of an antigen that is bound by an antibody is termed the “epitope”. This interaction depends on the presence of structural, hydrophobic/hydrophilic, and/or electrostatic features that allow appropriate chemical or molecular interactions between the molecules. Thus an antibody is commonly said to “bind” (or “specifically bind”) or be “reactive with” (or “specifically reactive with”), or, equivalently, “reactive against” (or “specifically reactive against”) the epitope of its target antigen. Antibodies are commonly described in the art as being “against” or “to” their antigens as shorthand for antibody binding to the antigen. Thus an “antibody that binds LPA,” an “antibody reactive against LPA,” an “antibody reactive with LPA,” an “antibody to LPA,” and an “anti-LPA antibody” all have the same meaning. Antibody molecules can be tested for specificity of binding by comparing binding to the desired antigen to binding to unrelated antigen or analogue antigen or antigen mixture under a given set of conditions. Preferably, an antibody used according to the invention will lack significant binding to unrelated antigens, or even analogs of the target antigen.

“Stem cells” are undifferentiated cells capable of renewing themselves through cell division, sometimes after long periods of inactivity. Second, under certain physiologic or experimental conditions, they can be induced to differentiate into tissue- or organ-specific cells with special functions. Types of stem cells include embryonic stem cells (ESCs), adult stem cells (ASCs), umbilical cord stem cells and induced pluripotent stem cells (iPSCs) which are somatic (adult) cells reprogrammed to enter an embryonic stem cell-like state.

“Stem cell therapy” or “stem cell transplant” refers to the infusion of healthy stem cells into the body. These may be cells which are from a donor or other source (heterologous transplant) or the recipient's own cells (autologous transplant). In the latter case the recipient's cells may be treated, for example with gene therapy, siRNA, antisense or other treatment to correct a defect before re-introduction into the body.

The “stem cell therapeutic window” refers to a time period during which an inhibitor of LPA has a positive effect on the outcome of stem cell therapy.

A “subject” or “patient” refers to an animal in need of treatment that can be effected by molecules of the invention. Animals that can be treated in accordance with the invention include vertebrates, with mammals such as bovine, canine, equine, feline, ovine, porcine, and primate (including humans and non-human primates) animals being particularly preferred examples. A “stem cell therapy subject” or “stem cell therapy patient” is a subject or patient undergoing, having undergone, or about to undergo stem cell therapy.

A “therapeutic agent” refers to a drug or compound that is intended to provide a therapeutic effect including, but not limited to: anti-inflammatory drugs including COX inhibitors and other NSAIDS, anti-angiogenic drugs, chemotherapeutic drugs as defined above, cardiovascular agents, immunomodulatory agents, agents that are used to treat neurodegenerative disorders, opthalmic drugs, etc.

A “therapeutically effective amount” (or “effective amount”) refers to an amount of an active ingredient, e.g., an agent according to the invention, sufficient to effect treatment when administered to a subject in need of such treatment. Accordingly, what constitutes a therapeutically effective amount of a composition according to the invention may be readily determined by one of ordinary skill in the art. For example, in the context of cancer therapy, a “therapeutically effective amount” is one that produces an objectively measured change in one or more parameters associated with cancer cell survival or metabolism, including an increase or decrease in the expression of one or more genes correlated with the particular cancer, reduction in tumor burden, cancer cell lysis, the detection of one or more cancer cell death markers in a biological sample (e.g., a biopsy and an aliquot of a bodily fluid such as whole blood, plasma, serum, urine, etc.), induction of induction apoptosis or other cell death pathways, etc. Of course, the therapeutically effective amount will vary depending upon the particular subject and condition being treated, the weight and age of the subject, the severity of the disease condition, the particular compound chosen, the dosing regimen to be followed, timing of administration, the manner of administration and the like, all of which can readily be determined by one of ordinary skill in the art. It will be appreciated that in the context of combination therapy, what constitutes a therapeutically effective amount of a particular active ingredient may differ from what constitutes a therapeutically effective amount of that active ingredient when administered as a monotherapy (i.e., a therapeutic regimen that employs only one chemical entity as the active ingredient).

As used herein, the terms “therapy” and “therapeutic” encompasses the full spectrum of prevention and/or treatments for a disease, disorder or physical trauma. A “therapeutic” agent of the invention may act in a manner that is prophylactic or preventive, including those that incorporate procedures designed to target individuals that can be identified as being at risk (pharmacogenetics); or in a manner that is ameliorative or curative in nature; or may act to slow the rate or extent of the progression of at least one symptom of a disease or disorder being treated; or may act to minimize the time required, the occurrence or extent of any discomfort or pain, or physical limitations associated with recuperation from a disease, disorder or physical trauma; or may be used as an adjuvant to other therapies and treatments.

The term “transdifferentiation” means the direct conversion of one mature (differentiated) cell phenotype to another.

The term “treatment” or “treating” means any treatment of a disease or disorder, including preventing or protecting against the disease or disorder (that is, causing the clinical symptoms not to develop); inhibiting the disease or disorder (i.e., arresting, delaying or suppressing the development of clinical symptoms; and/or relieving the disease or disorder (i.e., causing the regression of clinical symptoms). As will be appreciated, it is not always possible to distinguish between “preventing” and “suppressing” a disease or disorder because the ultimate inductive event or events may be unknown or latent. Those “in need of treatment” include those already with the disorder as well as those in which the disorder is to be prevented. Accordingly, the term “prophylaxis” will be understood to constitute a type of “treatment” that encompasses both “preventing” and “suppressing”. The term “protection” thus includes “prophylaxis”.

The term “therapeutic regimen” means any treatment of a disease or disorder using chemotherapeutic and cytotoxic agents, radiation therapy, surgery, gene therapy, DNA vaccines and therapy, siRNA therapy, anti-angiogenic therapy, immunotherapy, bone marrow transplants, aptamers and other biologics such as antibodies and antibody variants, receptor decoys and other protein-based therapeutics.

The term “variable” region (of an antibody) comprises framework and complementarity regions or CDRs (otherwise known as hypervariable regions) refers to certain portions of the variable domains that differ extensively in sequence among antibodies and are used in the binding and specificity of each particular antibody for its particular antigen. However, the variability is not evenly distributed throughout the variable domains of antibodies. It is concentrated in three segments called hypervariable regions (CDRs) both in the light chain and the heavy chain variable domains. The more highly conserved portions of variable domains are called the framework region (FR). The variable domains of native heavy and light chains each comprise four FRs (FR1, FR2, FR3 and FR4, respectively), largely adopting a β-sheet configuration, connected by three hypervariable regions, which form loops connecting, and in some cases forming part of, the beta-sheet structure. The term “hypervariable region” when used herein refers to the amino acid residues of an antibody which are responsible for antigen binding. The hypervariable region comprises amino acid residues from a “complementarity determining region” or “CDR” (for example residues 24-34 (L1), 50-56 (L2) and 89-97 (L3) in the light chain variable domain and 31-35 (H1), 50-65 (H2) and 95-102 (H3) in the heavy chain variable domain; Kabat, et al., Sequences of Proteins of Immunological Interest, 5th Ed. Public Health Service, National Institutes of Health, Bethesda, Md. (1991)) and/or those residues from a “hypervariable loop” (for example residues 26-32 (L1), 50-52 (L2) and 91-96 (L3) in the light chain variable domain and 26-32 (H1), 53-55 (H2) and 96-101 (H3) in the heavy chain variable domain; Chothia and Lesk, J. Mol. Biol. 196:901-917 (1987)). “Framework” or “FR” residues are those variable domain residues other than the hypervariable region residues as herein defined. The hypervariable regions in each chain are held together in close proximity by the FRs and, with the hypervariable regions from the other chain, contribute to the formation of the antigen-binding site of antibodies (see Kabat, et al., above, pages 647-669). Thus the uniqueness of an antibody for binding its antigen comes from the CDRs (hypervariable regions) and their arrangement in space, rather than the particular framework which holds them there. The CDRs can be placed into any of a variety of frameworks as long as a desired level of antigen binding is retained. The constant domains are not involved directly in binding an antibody to an antigen, but exhibit various effector functions, such as participation of the antibody in antibody-dependent cellular toxicity.

### SUMMARY OF THE INVENTION

The instant invention provides methods of stem cell therapy comprising delivering to a stem cell therapy subject during a stem cell therapeutic window an inhibitor of lysophosphatidic acid (LPA), thereby effecting stem cell therapy. The inhibitor of LPA may be a direct inhibitor of LPA, e.g., an agent that reduces the activity or effective concentration of lysophosphatidic acid, such as an agent that binds and neutralizes lysophosphatidic acid. A preferred inhibitor of LPA is an anti-LPA antibody, preferably a humanized antibody. The inhibitor of LPA may be an indirect inhibitor of lysophosphatidic acid, e.g. an agent that inhibits LPA action on receptors, inhibits LPA biosynthesis or stimulates LPA degradation, such as an LPA receptor antagonist, an inhibitor of LPA biosynthesis, an LPA-degrading enzyme or an activator or agonist of an LPA-degrading enzyme. A preferred indirect inhibitor of LPA is an autotaxin inhibitor. Also provided are methods of preparing stem cells for use in stem cell therapy, comprising culturing said stem cells in the presence of an LPA inhibitor.

These and other aspects and embodiments of the invention are discussed in greater detail in the sections that follow. As those in the art will appreciate, the following description describes certain preferred embodiments of the invention in detail, and is thus only representative and does not depict the actual scope of the invention. Before describing the present invention in detail, it is understood that the invention is not limited to the particular molecules, systems, and methodologies described, as these may vary. It is also to be understood that the terminology used herein is for the purpose of describing particular embodiments only, and is not intended to limit the scope of the invention defined by the appended claims.

### BRIEF DESCRIPTION OF THE DRAWING

This application contains one FIGURE executed in color. Copies of this application with color drawing will be provided upon request and payment of the necessary fee. A brief summary of the FIGURE is provided below.

FIG. 1 is a micrograph showing mouse brains after cortical injury. Panel A on the left shows a mouse brain with an area of hemorrhage as typically seen after TBI in the cortical impact model. Panel B on the right shows a mouse brain after TBI in the same model, but treated with anti-LPA antibody. The hemorrhage normally observed in this model is greatly reduced.

### DETAILED DESCRIPTION OF THE INVENTION

The present invention relates to methods for stem cell therapy that incorporate inhibition of lysophosphatidic acid (LPA).

1. Inhibitors of LPA

Inhibitors of LPA are agents that interfere with LPA activity or lower the effective concentration of LPA, typically but not necessarily under physiological conditions. In different embodiments of the invention, LPA activity may be blocked by direct and indirect methods.

A. Direct Inhibitors of LPA:

Direct methods include those involving agents that directly bind to and inhibit the activity or effective concentration of LPA. Such agents include but are not limited to antibodies or antibody-like molecules (e.g. DARPins), LPA receptor fragments or decoys, and aptamers.

i. Antibodies

Antibody molecules or immunoglobulins are large glycoprotein molecules with a molecular weight of approximately 150 kDa, usually composed of two different kinds of polypeptide chain. One polypeptide chain, termed the heavy chain (H) is approximately 50 kDa. The other polypeptide, termed the light chain (L), is approximately 25 kDa. Each immunoglobulin molecule usually consists of two heavy chains and two light chains. The two heavy chains are linked to each other by disulfide bonds, the number of which varies between the heavy chains of different immunoglobulin isotypes. Each light chain is linked to a heavy chain by one covalent disulfide bond. In any given naturally occurring antibody molecule, the two heavy chains and the two light chains are identical, harboring two identical antigen-binding sites, and are thus said to be divalent, i.e., having the capacity to bind simultaneously to two identical molecules.

The light chains of antibody molecules from any vertebrate species can be assigned to one of two clearly distinct types, kappa (k) and lambda (λ), based on the amino acid sequences of their constant domains. The ratio of the two types of light chain varies from species to species. As a way of example, the average k to λ ratio is 20:1 in mice, whereas in humans it is 2:1 and in cattle it is 1:20.

The heavy chains of antibody molecules from any vertebrate species can be assigned to one of five clearly distinct types, called isotypes, based on the amino acid sequences of their constant domains. Some isotypes have several subtypes. The five major classes of immunoglobulin are immunoglobulin M (IgM), immunoglobulin D (IgD), immunoglobulin G (IgG), immunoglobulin A (IgA), and immunoglobulin E (IgE). IgG is the most abundant isotype and has several subclasses (IgG1, 2, 3, and 4 in humans). The Fc fragment and hinge regions differ in antibodies of different isotypes, thus determining their functional properties. However, the overall organization of the domains is similar in all isotypes.

Of note, variability is not uniformly distributed throughout the variable domains of antibodies, but is concentrated in three segments called complementarity-determining regions (CDRs) or hypervariable regions, both in the light-chain and the heavy-chain variable domains. The more highly conserved portions of variable domains are called the framework region (FR). The variable domains of native heavy and light chains each comprise four FR regions connected by three CDRs. The CDRs in each chain are held together in close proximity by the FR regions and, with the CDRs from the other chain, contribute to the formation of the antigen-binding site of antibodies (see Kabat, et al., Sequences of Proteins of Immunological Interest, Fifth Edition, National Institute of Health, Bethesda, Md. (1991)). Collectively, the 6 CDRs contribute to the binding properties of the antibody molecule. However, even a single variable domain (or half of an Fv comprising only three CDRs specific for an antigen) has the ability to recognize and bind antigen (see Pluckthun, in The Pharmacology of Monoclonal Antibodies, vol. 113, Rosenburg and Moore, eds., Springer-Verlag, New York, pp. 269-315 (1994)).

The constant domain refers to the C-terminal region of an antibody heavy or light chain. Generally, the constant domains are not directly involved in the binding properties of an antibody molecule to an antigen, but exhibit various effector functions, such as participation of the antibody in antibody-dependent cellular toxicity. Here, “effector functions” refer to the different physiological effects of antibodies (e.g., opsonization, cell lysis, mast cell, basophil and eosinophil degranulation, and other processes) mediated by the recruitment of immune cells by the molecular interaction between the Fc domain and proteins of the immune system. The isotype of the heavy chain determines the functional properties of the antibody. Their distinctive functional properties are conferred by the carboxy-terminal portions of the heavy chains, where they are not associated with light chains.

Antibody molecules can be tested for specificity of antigen binding by comparing binding to the desired antigen to binding to unrelated antigen or analogue antigen or antigen mixture under a given set of conditions. Preferably, an antibody according to the invention will lack significant binding to unrelated antigens, or even analogs of the target antigen.

The term “antibody,” in the context of this invention, is used in the broadest sense, and encompasses monoclonal, polyclonal, multispecific (e.g., bispecific, wherein each arm of the antibody is reactive with a different epitope of the same or different antigen), minibody, heteroconjugate, diabody, triabody, chimeric, and synthetic antibodies, as well as antibody fragments, derivatives and variants that specifically bind an antigen with a desired binding property and/or biological activity.

Desired activities can include the ability to bind the antigen specifically, the ability to inhibit proleration in vitro, the ability to inhibit angiogenesis in vivo, and the ability to alter cytokine profile(s) in vitro.

Native antibodies (native immunoglobulins) are usually heterotetrameric glycoproteins of about 150,000 Daltons, typically composed of two identical light (L) chains and two identical heavy (H) chains. Each light chain is typically linked to a heavy chain by one covalent disulfide bond, while the number of disulfide linkages varies among the heavy chains of different immunoglobulin isotypes. Each heavy and light chain also has regularly spaced intrachain disulfide bridges. Each heavy chain has at one end a variable domain (V_{H}) followed by a number of constant domains. Each light chain has a variable domain at one end (V_{L}) and a constant domain at its other end; the constant domain of the light chain is aligned with the first constant domain of the heavy chain, and the light-chain variable domain is aligned with the variable domain of the heavy chain. Particular amino acid residues are believed to form an interface between the light- and heavy-chain variable domains.

The light chains of antibodies (immunoglobulins) from any vertebrate species can be assigned to one of two clearly distinct types, called kappa (κ) and lambda (λ), based on the amino acid sequences of their constant domains.

Depending on the amino acid sequence of the constant domain of their heavy chains, immunoglobulins can be assigned to different classes. There are five major classes of immunoglobulins: IgA, IgD, IgE, IgG, and IgM, and several of these may be further divided into subclasses (isotypes), e.g., IgG1, IgG2, IgG3, IgG4, IgA, and IgA2. The heavy-chain constant domains that correspond to the different classes of immunoglobulins are called alpha, delta, epsilon, gamma, and mu, respectively. The subunit structures and three-dimensional configurations of different classes of immunoglobulins are well known.

a. Antibodies to LPA

A polyclonal antibody to LPA is described in Chen, et al., Bioorg Med Chem Lett, 2000 Aug. 7; 10(15):1691-3). Monoclonal antibodies to LPA are described in Sabbadini, et al., U.S. patent application publication no. 20080145360, published Jun. 19, 2008 (attorney docket no. LPT-3100-UT4), and U.S. patent application publication no. 20090136483 (attorney docket no. LPT-3200-UT), published May 28, 2009, both of which are herein incorporated by reference in their entirety for all purposes. The former publication describes the production and characterization of a series of murine monoclonal antibodies against LPA and the latter publication describes a humanized monoclonal antibody against LPA. Additional humanized monoclonal antibodies against LPA are disclosed in U.S. patent application Ser. No. 12/761,584, filed Apr. 16, 2010 (attorney docket no. LPT-3210-UT), the contents of which are also incorporated herein in their entirety. The specificity of several murine antibodies for various LPA isoforms is shown in Table 1, below. IC₅₀: Half maximum inhibition concentration; MI: Maximum inhibition (% of binding in the absence of inhibitor); - - - : not estimated because of weak inhibition. A high inhibition result indicates recognition of the competitor lipid by the antibody.

TABLE 1
Specificity profile of six anti-LPA mAbs [from U.S. Pub. No. 20080145360]

| | 14:0 LPA | | 16:0 LPA | | 18:1 LPA | | 18:2 LPA | | 20:4 LPA | |
|---|---|---|---|---|---|---|---|---|---|---|
| | IC₅₀ | MI | IC₅₀ | MI | IC₅₀ | MI | IC₅₀ | MI | IC₅₀ | MI |
| | uM | % | uM | % | uM | % | uM | % | uM | % |
| B3 | 0.02 | 72.3 | 0.05 | 70.3 | 0.287 | 83 | 0.064 | 72.5 | 0.02 | 67.1 |
| B7 | 0.105 | 61.3 | 0.483 | 62.9 | >2.0 | 100 | 1.487 | 100 | 0.161 | 67 |
| B58 | 0.26 | 63.9 | 5.698 | >100 | 1.5 | 79.3 | 1.240 | 92.6 | 0.304 | 79.8 |
| B104 | 0.32 | 23.1 | 1.557 | 26.5 | 28.648 | >100 | 1.591 | 36 | 0.32 | 20.1 |
| D22 | 0.164 | 34.9 | 0.543 | 31 | 1.489 | 47.7 | 0.331 | 31.4 | 0.164 | 29.5 |
| A63 | 1.147 | 31.9 | 5.994 | 45.7 | — | — | — | — | 0.119 | 14.5 |
| B3A6 | 0.108 | 59.9 | 1.151 | 81.1 | 1.897 | 87.6 | — | — | 0.131 | 44.9 |

Interestingly, the anti-LPA mAbs were able to discriminate between 12:0 (lauroyl), 14:0 (myristoyl), 16:0 (palmitoyl), 18:1 (oleoyl), 18:2 (linoleoyl), and 20:4 (arachidonoyl) LPAs. A desirable EC₅₀ rank order for ultimate drug development is 18:2>18:1>20:4 for unsaturated lipids and 14:0>16:0>18:0 for the saturated lipids, along with high specificity. The specificity of the anti-LPA mAbs was assessed for their binding to LPA-related biolipids such as distearoyl-phosphatidic acid, lysophosphatidylcholine, S1P, ceramide, and ceramide-1-phosphate. None of the anti-LPA antibodies demonstrated cross-reactivity to distearoyl PA and LPC, the immediate metabolic precursor of LPA.

Biophysical Properties of B7 Antibody

The anti-LPA monoclonal antibody B7 has high affinity for the signaling lipid LPA (K_{D} of 1-50 pM as demonstrated by surface plasmon resonance in the BiaCore assay, and in a direct binding ELISA assay); in addition, B7 demonstrates high specificity for LPA, having shown no binding affinity for over 100 different bioactive lipids and proteins, including over 20 bioactive lipids, some of which are structurally similar to LPA. The murine antibody is a full-length IgG1k isotype antibody composed of two identical light chains and two identical heavy chains with a total molecular weight of 155.5 kDa. The biophysical properties are summarized in Table 2, below.

TABLE 2
General Properties of Monoclonal Antibody B7

| | |
|---|---|
| Identity | B7 |
| Antibody isotype | Murine IgG1k |
| Specificity | Lysophosphatidic acid (LPA) |
| Molecular weight | 155.5 kDa |
| OD of 1 mg/mL | 1.35 (solution at 280 nm) |
| K_{D} | 1-50 pM |
| Apparent Tm | 67° C. at pH 7.4 |
| Appearance | Clear if dissolved in 1x PBS buffer (6.6 mM |
| | phosphate, 154 mM sodium chloride, pH 7.4) |
| Solubility | >40 mg/mL in 6.6 mM phosphate, 154 mM sodium |
| | chloride, pH 7.4 |

Lpathomab has also shown biological activity in preliminary cell based assays such as cytokine release, migration and invasion; these are summarized below along with data showing specificity of B7 for LPA isoforms and other bioactive lipids, and in vitro biological effects of B7.

TABLE 3
Biologic properties of Monoclonal Antibody B7
B7

| A. Competitor | 14:0 | 16:0 | 18:1 | 18:2 | 20:4 |
|---|---|---|---|---|---|
| Lipid | LPA | LPA | LPA | LPA | LPA |
| IC₅₀ (mM) | 0.105 | 0.483 | >2.0 | 1.487 | 0.161 |
| MI (%) | 61.3 | 62.9 | 100 | 100 | 67 |
| B. Competitor | | | | | |
| Lipid | LPC | S1P | C1P | Cer | DSPA |
| MI (%) | 0 | 2.7 | 1.0 | 1 | 0 |

| C. Cell based | LPA | % Inhibition |
|---|---|---|
| assay | isoform | (over LPA taken as 100) |
| Migration | 18:1 | 35* |
| Invasion | 14:0 | 95* |
| IL-8 Release | 18:1 | 20 |
| IL-6 Release | 18:1 | 23* |
| | | % Induction |
| | | (over LPA + TAXOL taken as 100) |
| Apoptosis | 18:1 | 79 |

A. Competition ELISA assay was performed with Lpathomab and 5 LPA isoforms. 18:1 LPA was captured on ELISA plates. Each competitor lipid (up to 10 mM) was serially diluted in BSA/PBS and incubated with 3 nM Lpathomab. Mixtures were then transferred to LPA coated wells and the amount of bound antibody was measured.
B. Competition ELISA was performed to assess specificity of Lpathomab. Data were normalized to maximum signal (A₄₅₀) and were expressed as percent inhibition (n = 3). IC₅₀: half maximum inhibition concentration; MI %: maximum inhibition (% of binding in the absence of inhibitor).
C. Migration assay: Lpathomab (150 mg/mL) reduced SKOV3 cell migration triggered by 1 mM LPA (n = 3); Invasion assay. Lpathomab (15 mg/mL) blocked SKOV3 cell invasion triggered by 2 mM LPA (n = 2); Cytokine release of human IL-8 and IL-6: Lpathomab (300-600 mg/mL, respectively) reduced 1 mM LPA-induced release of pro-angiogenic and metastatic IL-8 and IL-6 in SKOV3 conditioned media (n = 3). Apoptosis: SKOV3 cells were treated with 1 mM Taxol; 1 mM LPA blocked Taxol induced caspase-3 activation. The addition to Lpathomab (150 mg/mL) blocked LPA-induced protection from apoptosis (n = 1).
Data Analysis: Student-t test,
*denotes p < 0.05.

The potent and specific binding of B7 to LPA results in reduced availability of extracellular LPA with potentially therapeutic effects against cancer-, angiogenic- and fibrotic-related disorders.

A second murine anti-LPA antibody, B3, was also subjected to binding analysis as shown in Table 4, below.

TABLE 4
Biochemical characteristics of Monoclonal Antibody B3
Biochemical characteristics of B3 antibody

| | High density | Low density |
|---|---|---|
| A. BIACORE | surface | surface |
| Lipid Chip | 12:0 LPA | 18:0 LPA |
| K_{D} (pM), site 1 (site2) | 61 (32) | 1.6 (0.3) |
| B. Competition Lipid Cocktail | | |
| (C₁₆:C₁₈:C_{18:1}:C_{18:2}:C_{20:4}, | | |
| ratio 3:2:5:11:2) | (μM) | |
| IC50 | 0.263 | |
| C. Neutralization Assay | | |
| B3 antibody (nmol) | LPA (nmol) | |
| 0 | 0.16 | |
| 0.5 | 0.0428 | |
| 1 | 0.0148 | |
| 2 | under limit | |
| | of detection | |

A. Biacore analysis for B3 antibody. 12:0 and 18:0 isoforms of LPA were immobilized onto GLC sensor chips; solutions of B3 were passed over the chips and sensograms were obtained for both 12:0 and 18:0 LPA chips. Resulted sensograms showed complex binding kinetics of the antibody due to monovalent and bivalent antibody binding capacities. K_{D} values were calculated approximately for both LPA 12 and LPA 18.
B. Competition ELISA assay was performed with B3 and a cocktail of LPA isoforms (C₁₆:C₁₈:C_{18:1}:C_{18:2}:C_{20:4} in ratio 3:2:5:11:2). Competitor/Cocktail lipid (up to 10 μM) was serially diluted in BSA/PBS and incubated with 0.5 μg/mL B3. Mixtures were then transferred to a LPA coated well plate and the amount of bound antibody was measured. Data were normalized to maximum signal (A₄₅₀) and were expressed as IC₅₀ (half maximum inhibition concentration).
C. Neutralization assay: Increasing concentrations of B3 were conjugated to a gel. Mouse plasma was then activated to increase endogenous levels of LPA. Activated plasma samples were then incubated with the increasing concentrations of the antibody-gel complex. LPA leftover which did not complex to the antibody was then determined by ELISA. LPA was sponged up by B3 in an antibody concentration dependent way.

Humanization of B7

The variable domains of the B7 murine anti-LPA monoclonal antibody were humanized by grafting the murine CDRs into human framework regions (FR). See U.S. Provisional Patent Application 61/170,595, filed Apr. 17, 2009, the contents of which are herein incorporated by reference in their entirety for all purposes. For descriptions of CDR grafting techniques, see, for example, Lefranc, M. P, (2003). Nucleic Acids Res, 31: 307-10; Martin and Thornton (1996), J Mol Biol, 1996. 263: 800-15; Morea, et al. (2000), Methods, 20: 267-79; Foote and Winter (1992), J Mol Biol, 224: 487-99; Chothia, et al., (1985). J Mol Biol, 186:651-63.

Suitable acceptor human FR sequences were selected from the IMGT and Kabat databases based on a homology to B7 using a sequence alignment and analysis program (SR v 7.6). Lefranc (2003), supra; Kabat, et al. (1991), Sequences of Proteins of Immunological Interest, NIH National Techn. Inform. Service, pp. 1-3242. Sequences with high identity at FR, vernier, canonical and VH-VL interface residues (VCI) were initially selected. From this subset, sequences with the most non-conservative VCI substitutions, unusual proline or cysteine residues and somatic mutations were excluded. AJ002773 was thus selected as the human framework on which to base the humanized version of B7 heavy chain variable domain and DQ187679 was thus selected as the human framework on which to base the humanized version of B7 light chain variable domain.

A three-dimensional (3D) model containing the humanized VL and VH sequences was constructed to identify FR residues juxtaposed to residues that form the CDRs. These FR residues potentially influence the CDR loop structure and the ability of the antibody to retain high affinity and specificity for the antigen. Based on this analysis, 6 residues in AJ002773 and 3 residues in DQ187679 were identified, deemed significantly different from B7, and considered for mutation back to the murine sequence.

The sequence of the murine anti-LPA mAb B7 was humanized with the goal of producing an antibody that retains high affinity, specificity and binding capacity for LPA. Further, seven humanized variants were transiently expressed in HEK 293 cells in serum-free conditions, purified and then characterized in a panel of assays. Plasmids containing sequences of each light chain and heavy chain were transfected into mammalian cells for production. After 5 days of culture, the mAb titer was determined using quantitative ELISA. All combinations of the heavy and light chains yielded between 2-12 ug of antibody per ml of cell culture.

Characterization and Activity of the Humanized Variants

All the humanized anti-LPA mAb variants exhibited binding affinity in the low picomolar range similar to a chimeric anti-LPA antibody (also known as LT3010) and the murine antibody B7. All of the humanized variants exhibited a T_{M} similar to or higher than that of B7. With regard to specificity, the humanized variants demonstrated similar specificity profiles to that of B7. For example, B7 demonstrated no cross-reactivity to lysophosphatidyl choline (LPC), phosphatidic acid (PA), various isoforms of lysophosphatidic acid (14:0 and 18:1 LPA, cyclic phosphatidic acid (cPA), and phosphatidylcholine (PC).

Five humanized variants were further assessed in in vitro cell assays. LPA is important in eliciting release of interleukin-8 (IL-8) from cancer cells. B7 reduced IL-8 release from ovarian cancer cells in a concentration-dependent manner. The humanized variants exhibited a similar reduction of IL-8 release compared to B7.

Two humanized variants were also tested for their effect on microvessel density (MVD) in a Matrigel tube formation assay for neovascularization. Both were shown to decrease MVD formation.

Humanized Antibodies to LPA

LT3015 is a recombinant, humanized, monoclonal antibody that binds with high affinity to the bioactive lipid lysophosphatidic acid (LPA). LT3015 is a full-length IgG1k isotype antibody composed of two identical light chains and two identical heavy chains with a total molecular weight of 150 kDa. The heavy chain contains an N-linked glycosylation site. The two heavy chains are covalently coupled to each other through two intermolecular disulfide bonds, consistent with the structure of a human IgG1.

LT3015 was originally derived from the murine monoclonal antibody B7, which was produced using hybridomas generated from mice immunized with LPA. The humanization of the murine antibody involved the insertion of the six murine complementarity determining regions (CDRs) from murine antibody B7 in place of those of a human antibody framework selected for its structure similarity to the murine parent antibody. A series of substitutions were made in the framework to engineer the humanized antibody. These substitutions are called back mutations and replace human with murine residues that are involved in the interaction with the antigen. The final humanized version contains six murine back mutation in the human framework of variable domain of the heavy chain (pATH602) and three murine back mutations in the human framework of the variable domain of the light chain (pATH 502).

LT3114 is another recombinant, humanized, monoclonal antibody that binds with high affinity to the bioactive lipid lysophosphatidic acid (LPA). In contrast to LT3015, LT3114 was originally derived from the murine monoclonal antibody B3, meaning that the CDRs of LT3114 are identical to those of B3.

B. Indirect Inhibitors of LPA:

Indirect methods of inhibiting LPA signalling include those that employ agents that inhibit LPA action on receptors, inhibit LPA biosynthesis, or stimulate LPA degradation. Many of the indirect methods for inhibiting LPA activity have been described and are summarized in Tigyi (Br J Pharmacol. 2010 September; 161(2):241-70). There are between 5 and 7 receptors for LPA, including LPA1-5, GRP87, P2Y5, P2Y10, GRP35 and PPRgamma. Agents that are antagonists for one or more of these LPA receptors could be used in blocking LPA actions and would be useful in combination with stem cells to improve the effectiveness of stem cell therapy. Some specific examples of LPA receptor antagonists are: LPA1 specific antagonist is described by Swaney, et al. (J Pharmacol Exp Ther. 2011 March; 336(3):693-700), and an antagonist that blocks both LPA1 and LPA3 receptors has been described by Xu, et al. (J Med Chem. 2006 Aug. 24; 49(17):5309-15).

Another indirect approach to inhibiting LPA activity is inhibition of LPA biosynthesis. LPA is produced by one or more of the following enzymes: Autotaxin (ATX, Lyso PLD); PLA1, PLA2, MAG-kinase, and Glycerol-3 phosphate acyltransferase (GPAT). The most important enzyme in LPA generation is autotaxin, but atx +/−mice still have 50% blood LPA level. A specific autotaxin inhibitor is described by Gupte, et al., ChemMedChem. 2011 May 2; 6(5):922-35. This agent could be useful in promoting stem cell activity when co-administered with stem cells.

An additional indirect mode of anti-LPA action would be to stimulate endogenous degradative pathways for LPA, thus lowering the concentration or amount of LPA. There are several enzymatic pathways involved in LPA degradation, including LPA acyltransferase (LPATT), Lipid phosphatase (LPP), and other non-specific lysophospholipid lipases. Small molecule activators/agonists of these enzymes acting in a gain-of-function (GOF) mode would result in the desired lowering of LPA levels. Alternatively, one or more of these LPA degrading enzymes could be useful as biological agents and could themselves be administered to effect anti-LPA therapy, alone or as part of stem cell therapy.

3. Administration

a. Methods of Administration.

The stem cell therapy described herein can be achieved, in part, by administering LPA inhibitors by various routes employing different formulations and devices. Suitable pharmaceutically acceptable diluents, carriers, and excipients are well known in the art. One skilled in the art will appreciate that the amounts to be administered for any particular treatment protocol can readily be determined. Suitable amounts might be expected to fall within the range of 10 ug/dose to 10 g/dose, preferably within 10 mg/dose to 1 g/dose.

Drug substances may be administered by techniques known in the art, including but not limited to systemic, subcutaneous, intradermal, mucosal, including by inhalation, and topical administration. The mucosa refers to the epithelial tissue that lines the internal cavities of the body. For example, the mucosa comprises the alimentary canal, including the mouth, esophagus, stomach, intestines, and anus; the respiratory tract, including the nasal passages, trachea, bronchi, and lungs; the surface of the eye and the genitalia. Local administration (as opposed to systemic administration) may be advantageous because this approach can limit potential systemic side effects, but still allow therapeutic effect. Local administration also includes direct administration to the target tissue for the stem cell therapy, or to the fluid bathing said tissue. This may be by direct injection into tissues or fluid, by catheter or other means (e.g., cardiac catheterization).

For murine, pharmacokinetic and pharmacology studies, typically murine antibodies are used and are delivered intravenously (i.v.) However, antibodies can also be delivered by other routes, such as subcutaneously (s.c.) intraventricularly or intrathecally (i.th.). When administered s.c., anti-lipid antibodies have been shown to have excellent biodistribution (>80%) within several hours. Lpath and collaborators have good efficacy data in neuropathic pain models with both i.v. and intrathecal dosing.

4. Applications

The instant invention provides methods of stem cell therapy. These methods comprise administration of inhibitors of LPA in combination with stem cells, in order to reduce the effective concentration or activity of LPA in the vicinity of the stem cells. In one embodiment, the inhibitor of LPA is an agent, such as an antibody, that binds and neutralizes LPA. While not wanting to be bound by theory, it is generally believed that antibodies to LPA bind to LPA and sponge up (neutralize) LPA molecules, thus lowering the effective concentration of LPA. High concentrations of LPA are known to inhibit neuronal differentiation of NSCs. It is believed that interfering with LPA activity or lowering the effective concentration of LPA is useful in promoting differentiation, survival, engraftment and homing of stem cell transplants.

The instant invention also provides methods of preparing stem cells for transplantation into a subject, by culturing the stem cells in the presence of an inhibitor of LPA. Adding such an inhibitor has been shown to promote differentiation and survival of the stem cells and may also aid in engraftment and homing of the cells once administered to the patient.

Without wishing to be bound by any particular theory, it is believed that undesirably high concentrations of lipids such as LPA and/or its metabolites, which are sufficient to block differentiation of stem cells, may contribute to the development or symptomology of various diseases and disorders. Such diseases are believed to include neurological conditions, including traumatic brain injury, spinal cord injury, neurodegenerative diseases (including Parkinson's, Alzheimer's, and Huntington's diseases), in which there is a net loss of neurons due, e.g., to insufficient neuronal differentiation; stroke and other conditions such as hemorrhage in which blood contacts the CNS, and brain cancers. Reactive astrocytes and glioma can produce high levels of LPA. LPA does not stop glial differentiation from NSCs. Dottori, et al. (2008) Stem Cells, May; 26(5):1146-54. Epub 2008 Feb. 28. Thus it is believed that inhibiting LPA would increase the “take” or efficacy of stem cell therapy by blocking the negative effects of aberrant, excessive or unwanted effective concentrations of LPA on stem cells.

Other diseases or conditions in which stem cell therapy is believed to be useful, and in which the compositions and methods of the invention are believed to be useful in enhancing the efficacy of stem cell therapy, include bone diseases and conditions, including joint defects and injuries; neuromuscular diseases and conditions such as muscle damage, amyotrophic lateral sclerosis (ALS) and muscular dystrophy; cardiac diseases or conditions including myocardial infarct and heart failure; ischemic conditions including those of the heart; pancreatic disease or conditions including diabetes; neurological disease or conditions including traumatic brain injury, brain or spinal cord hemorrhage, spinal cord injury, stroke, and neurodegenerative disease, including Parkinson's disease, Alzheimer's disease, Huntington's disease, and neurodegenerative disorders of the gastrointestinal tract causing motility disorder; liver disease; pulmonary disorders; and diseases and conditions of the skin, hair and nails such as radiation injury, wounds and baldness.

### EXAMPLES

The invention will be further described by reference to the following detailed examples. These Examples are in no way to be considered to limit the scope of the invention in any manner. Examples 1-9 were conducted, and Example 10 will be conducted, in the laboratory of Dr. Alice Pebay (University of Melbourne and the O'Brien Institute, Melbourne, Australia).

### Example 1

### Neurosphere Formation, Treatment, and Differentiation

Neurospheres were formed and cultured as described in Dottori, M. et al. (2008), supra. Briefly, human embryonic stem cells (HES-2, HES-3, and HES-4, WiCell Research Institute, Madison Wis.) were cultured according to previously published methods. Neuronal induction using noggin was performed according to published methods and after growth and subculture, cells were grown as neurospheres in the presence of growth factors. Neurospheres could be plated on dishes coated with laminin or fibronectin. When plated onto laminin and cultured with neural basal medium (NBM, R&D Systems, Minneapolis Minn.), neurospheres typically differentiate into neurons. Quantitation of neuron-forming spheres (a measure of neuronal differentiation) was done by counting the number of neurospheres from which neuronal outgrowth was visible. Neurospheres that failed to attach to the plate were not counted.

Plated neurospheres were incubated in the presence or absence of LPA (Sigma Aldrich, St. Louis, Mo.) and/or antibody (concentrations shown) for 5 days. Dilutions of LPA were made in 0.1% fatty acid-free bovine serum albumin (final concentration 0.01% BSA).

### Example 2

### LPA Inhibits Neurosphere Formation and Neuronal Differentiation

As shown by Dottori et al., LPA inhibits the ability of NSC to form neurospheres, even in the presence of bFGF and EGF. Briefly, noggin-treated cells were incubated in the presence or absence of LPA while being subcultured in suspension in NBM with bFGF and EGF (20 ng/ml each) for 11-14 days. The number of neurospheres formed was counted and it was found that in the presence of 10 μM LPA, 13.47%±6.94% of cultures formed neurospheres, compared to 48.60%±8.15% for control cultures untreated with LPA. Dottori, M. et al. (2008), supra.

The effect of LPA on an additional differentiation step, the differentiation of NSC toward mature cells, was also measured. When plated on laminin in NBM, neurospheres typically differentiate into neurons, as assayed by visible neurons, elongated cell shape and/or positive staining for β-tubulin. Dottori, et al. observed the formation of elongated cells positive for β-tubulin in the untreated control cells, the NSC incubated in LPA did not differentiate into elongated cells, and there were few if any β-tubulin positive cells in the neurospheres. In general, neurospheres plated in the presence of 10 μM LPA did not give rise to neuronal cells.

### Example 3

### Anti-LPA Antibodies Block LPA Inhibition of Neurosphere Formation

Using the conditions used in Example 2 for LPA treatment alone, noggin-treated cells were incubated in the presence or absence of LPA while being subcultured in suspension in NBM with bFGF and EGF (20 ng/ml each) for 5-7 days. The number of neurospheres formed was counted and it was found that in the presence of 10 μM LPA, as before, neurosphere formation was decreased (n≧3). Whereas control cells yielded 90.482±5.346% neurosphere formation, cells treated with 10 μM LPA yielded only 13.500±5.590% neurosphere formation. Cells treated with LPA at 1 μM, in contrast, yielded 50±12.50% neurosphere formation. Anti-LPA antibody B3 alone gave neurosphere formation comparable to control (91.667±8.333% for 0.1 mg/ml B3 and 91.667±4.167% at 1.0 mg/ml B3). Notably, the combination of 1 mg/ml B3 and 10 μM LPA also gave neurosphere formation comparable to control (95.833±4.167%), indicating that the antibody to LPA had blocked inhibition of neurosphere formation that normally occurs in the presence of LPA.

The size of the neurospheres was also measured after LPA+/−B3 antibody treatment (n=3 for each) under the same conditions as above. The neurosphere area after treatment with B3 antibody alone was 93.94%±3.61% of untreated control; neurosphere area after treatment with LPA+B3 was 75.18%±9.89% of control. Measurements after treatment with LPA alone were not possible because neurospheres do not form. Statistics indicate the variation in size between the treatment groups is not significant.

The data show that blocking LPA (from endogenous production by NSCs) does not significantly increase neurosphere size, and more importantly, that the effect of LPA on the growth of neurospheres is totally abolished by B3 (ie the size is normal and comparable to control): this reveals the potency of B3 in blocking LPA activity.

### Example 4

### Humanized and Murine Anti-LPA Antibodies Block LPA Inhibition of Neuronal Differentiation

Using the same conditions used in Example 2 for LPA treatment alone, plated neurospheres were treated with 10 μM LPA alone, or with anti-LPA antibody B3 or B7 (1 mg/ml) alone, or with 10 μM LPA in combination with 1 mg/ml of antibody B3 or B7. Similarly, cells were treated with 10 μM LPA alone, humanized anti-LPA antibody LT3015 alone (1 mg/ml) or with 10 μM LPA in combination with 1 mg/ml LT3015. The percent of neuron-forming neurospheres was quantitated as in Example 2 (beta-tubulin staining and quantification of neuron-forming spheres, as described in Dottori, et al (2008)). LPA alone reduced neuron-forming neurospheres to approximately 25.00±6.45% of untreated control. Neurosphere samples treated with B3 antibody alone had neuron-forming neurospheres equivalent to control (100%). Neurospheres treated with the combination of LPA and B3 antibody had neuron forming neurospheres equal to 86.66±5.65% of control, indicating that the antibody had blocked the inhibition of neuron formation that normally occurs in the presence of LPA. Cells treated with the combination of LPA and LT3015 humanized antibody showed nearly identical neuron formation to B3-treated cells (87.5%±12.50% of control). The antibody B7, under similar conditions, had little to no effect in this experiment (37.00±5.31% of control).

### Example 5

### Humanized and Murine Anti-LPA Antibodies Block LPA Inhibition of Neurosphere Formation

Using the conditions described in previous examples, HSC were plated onto laminin for neuronal differentiation in NBM medium (3 days), with or without LPA (10 μM), with or antibody to LPA at 1 mg/ml (B3, B7, or the humanized antibody LT3015, tested singly with or without LPA).

As before, the number of neuron-forming spheres was significantly decreased in the presence of 10 μM LPA, to approximately 26% of control. None of the antibodies when tested alone had any effect on number of neuron-forming spheres (all were equivalent to control, which was 100%). However, all of the anti-LPA antibodies were able to block the inhibition of neuronal differentiation by LPA. Cells treated with B3 and LPA or with LT3015 and LPA had neuron-forming neurospheres equal to 75% of control. Cells treated with B7 and LPA had neuron-forming neurospheres equal to 50% of control. Pool of data results are similar: LPA 25.00±6.45%; B3+LPA: 86.66±5.65; B7+LPA 37.00±5.31%; Humanized B7 (LT3015): 87.5±12.5 (however although differentiation occurs there are fewer neurons observed than with B3) n=2 for hB7 and n>3 for B3 and B7. Thus, all three LPA antibodies, including LT3015, a humanized antibody to LPA, inhibit LPA's effect on neuronal differentiation, as measured by neurosphere formation. It was noted that neurospheres from cells treated with B3 and LPA had the greatest number of neurons (indicating further differentiation), followed by neurospheres from LT3015-treated cells, with a lesser number of neurons in neurospheres from cells treated with B7 antibody.

### Example 6

### Immunohistochemical Staining of LPA Using Monoclonal Antibody to LPA

Immunohistochemical methods can be used to determine the presence and location of LPA in cells. Spinal cords (adult (3 months old) male C57BL/6 mice) from animals with and without spinal cord injury were immunostained 4 days after injury. Adult C57BL/6 mice (20-30 g) were anaesthetized with a mixture of ketamine and xylazine (100 mg/kg and 16 mg/kg, respectively) in phosphate buffered saline (PBS) injected intraperitoneally. The spinal cord was exposed at the low thoracic to high lumbar area, at level T12, corresponding to the level of the lumbar enlargement. Fine forceps were used to remove the spinous process and lamina of the vertebrae and a left hemisection was made at T12. A fine scalpel was used to cut the spinal cord, which was cut a second time to ensure that the lesion was complete, on the left side of the spinal cord, and the overlying muscle and skin were then sutured. This resulted in paralysis of the left hindlimb. After 2 or 4 days the animals were re-anaesthetized as above and then perfused with PBS through the left ventricle of the heart, followed by 4% paraformaldehyde (PFA). After perfusion, the spinal cords were gently removed using fine forceps and post-fixed for 1 hour in cold 4% PFA followed by paraffin embedding or cryo-preserving in 20% sucrose in PBS overnight at 4° C. for frozen sections. Tissues for taken from n=3 uninjured mice and n=3 injured mice at 2 and 4 days post-injury. As described in Goldshmit Y, Galea M P, Wise G, Bartlett P F, Turnley A M: Axonal regeneration and lack of astrocytic gliosis in EphA4-deficient mice. J Neurosci 2004, 24(45):10064-10073.

IHC frozen spinal cord sagittal sections (10 μm) were examined using standard immunohistochemical procedures to determine expression and localization of the different LPA receptors. Frozen sections were postfixed for 10 min with 4% PFA and washed 3 times with PBS before blocking for 1 hour at room temperature (RT) in blocking solution containing 5% goat serum (Millipore) and 0.1% Triton-X in PBS in order to block non-specific antisera interactions. Primary antibodies used were B3 (0.1 mg/ml) rabbit anti-LPA₁ (1:100, Cayman Chemical, USA), rabbit anti-LPA₂ (1:100, Abcam, UK) and mouse anti-GFAP (1:500, Dako, Denmark). Primary antibodies were added in blocking solution and sections incubated over night at 4° C. They were then washed and incubated in secondary antibody for 1 hr at RT, followed by Dapi counterstain. Sections were coverslipped in Fluoromount (Dako) and examined using an Olympus BX60 microscope with a Zeiss Axiocam HRc digital camera and Zeiss Axiovision 3.1 software capture digital images. Some double labeled sections were also examined using a Biorad MRC1024 confocal scanning laser system installed on a Zeiss Axioplan 2 microscope. All images were collated and multi-colored panels produced using Adobe Photoshop 6.0.

After injury, non-neuronal glial cells in the CNS called astrocytes respond to many damage and disease states resulting in a “glial response”. Glial Fibrillary Acidic Protein (GFAP) antibodies are widely used to see the reactive astrocytes which form part of this response, since reactive astrocytes stain much more strongly with GFAP antibodies than normal astrocytes. LPA was revealed by immunohistochemistry using antibody B3 (0.1 mg/ml overnight). Fluorescence microscopy showed that reactive astrocytes are present in spinal cords 4 days after injury, and these cells stain positively for LPA. In contrast, uninjured (control) spinal cords have little to no staining for astrocytes or LPA. Thus LPA is present in reactive astrocytes of the spinal cord. In both injured and control animals, the central canal (hypothesized to be a stem cell niche) does not stain for LPA.

### Example 7

### Immunohistochemical Confirmation that Anti-LPA Antibodies Block LPA Inhibition of Neuronal Differentiation

Neurospheres grown and treated as in above examples were immunostained for CD133 ( 1/1000, Abcam, Inc., Cambridge Mass.), β-tubulin ( 1/500, Millipore, Billerica Mass.) or LPA (0.1 mg/ml) as described in the previous example. β-tubulin staining is indicative of differentiation of neurons. In contrast, CD133 staining is lost upon differentiation. With LPA treatment, CD133-positive cells are observed as the cells migrating out of the neurosphere. In control cells, the migrating cells are either weakly CD133 positive or are negative for CD133 staining. Expression of CD133 was seen to be reduced by the LPA antibodies (not quantitated).

### Example 8

### Anti-LPA Antibody in Murine Cortical Impact Model of Traumatic Brain Injury (TBI)

The mouse is an ideal model organism for TBI studies because there is an accepted model of human TBI, the type I IFN system in the mouse is similar to that in human, and the ability to generate gene-targeted mice helps to clarify cause and effect rather than mere correlations. Adult mice were anaesthetised with a single ip injection of Ketamine/Xylazine and the scalp above the parietal bones shaved with clippers. Each scalp was disinfected with chlorhexideine solution and an incision made to expose the right parietal bone. A dentist's drill with a fine burr tip was then used to make a 3 mm diameter circular trench of thinned bone centred on the centre of the right parietal bone. Fine forceps were then used to twist and remove the 3 mm plate of parietal bone to expose the parietal cortex underneath. The plate of bone removed was placed into sterile saline and retained. The mouse was mounted in a stereotaxic head frame and the tip of the impactor (2 mm diameter) positioned in the centre of the burr hole at right angles to the surface of the cortex and lowered until it just touches the dura mater membrane covering the cortex. A single impact injury (1.5 mm depth) was applied using the computer controller. The mouse was removed from the head frame and the plate of bone replaced. Bone wax was applied around the edges of the plate to seal and hold the plate in position. The skin incision was then closed with fine silk sutures and the area sprayed with chlorhexideine solution. The mouse was then returned to a holding box underneath a heat lamp and allowed to regain consciousness (total time anaesthetised=30-40 minutes).

Treatments:

Treatments or isotype controls were injected at various time points, either before or after TBI. Anti-LPA antibody (B3 or other) was injected by tail-IV (0.5 mg). Following 24-48 hours, the animals were sacrificed and their brains analysed.

Analysis:

Neuronal death/survival (TUNEL analysis), reactive astrogliosis (revealed by Ki67 positive cells co-labelled with GFAP) and NS/PC responses (proliferation by CD133/Ki67, migration to the injury site by CD133 and differentiation) are analysed. The immune response is assessed by CD11b immunostaining. Quantification is performed by density measurement using ImageJ (NIH).

Preliminary Results:

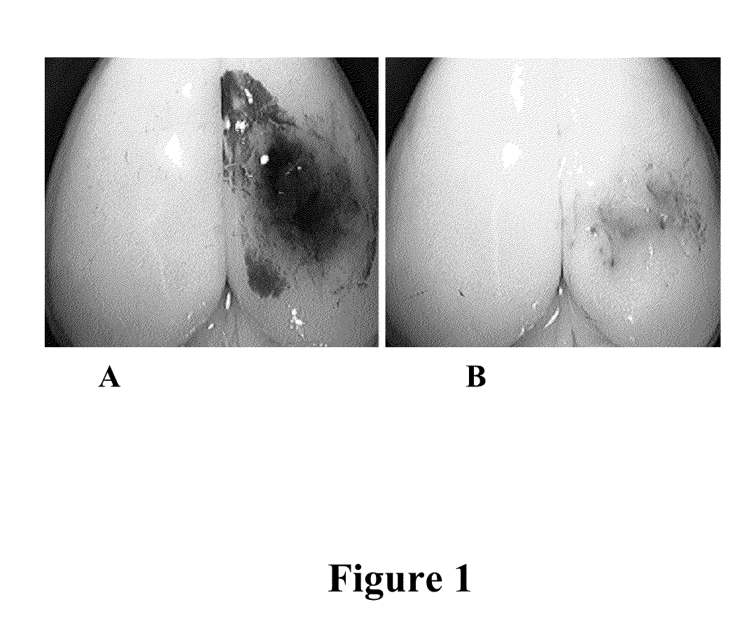

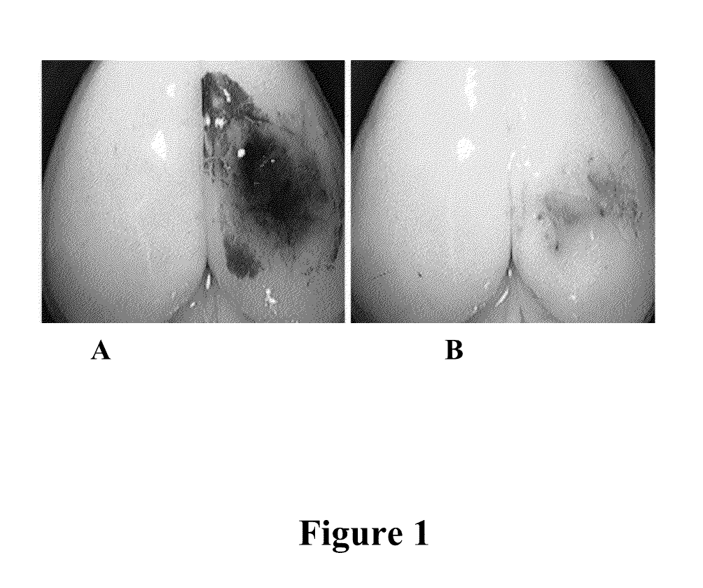

Preliminary data in this model show that anti-LPA antibody treatment (B3) reduces the degree of hemorrhage normally seen in the mouse brain following TBI in this cortical impact model (FIG. 1).

### Example 9

### LPA Inhibits the Neuronal Differentiation of Adult Mouse NSC

In mouse adult neurospheres generated from mouse subventricular zone NSC, expression analysis of the LPA receptors indicated the presence of the mRNA transcripts for LPA receptors LPA₁, LPA₃ and LPA₄ and absence or low level expression of the mRNA transcripts for LPA receptors LPA₂ and LPA₅, indicating that adult mNS/PC are also potential targets for LPA. Contrary to what was observed in human NSC, LPA did not modify neurosphere formation or growth of mouse NSC. However, and similarly to data obtained in human NSC, LPA inhibited the neuronal differentiation of adult mouse NSC by maintaining them as NSC when plated in conditions normally inducing neuronal differentiation. After three days, LPA (10 μM)-treated mouse NSC only showed low levels of expression of βIII-tubulin, a marker for differentiated neurons (26.25±2.08% of total cells), and remained mainly positive for nestin, a marker for undifferentiated NSCs (87.55±3.20% of total cells). In contrast, untreated cells showed greater levels of differentiated neurons (βIII-tubulin expressed by 57.12±18.42% of cells) and lower levels of undifferentiated NSCs (nestin was expressed by 58.01±6.20 of total cells). These effects were independent of apoptosis or proliferation.

### Example 10

### Combination of Anti-LPA Antibody and Stem Cell Transplantation

The impact of blocking LPA signaling on stem cell transplantation in the CNS is studied using a rodent model of spinal cord injury. Following injury, anti-LPA monoclonal antibodies are injected together with human NS/PC (derived from iPSC or ESC; prefereably GFP-expressing stem cells will be used). It is anticipated that antibody treatment coincident with stem cell transplant will improve the outcome of stem cell transplantation. The effects of anti-LPA antibody co-administration with stem cells in the uninjured and injured spinal cord will be evaluated to identify effects on NS/PC grafting and differentiation and to identify effects on host cells (neuronal response, glial scar, immune response) as well as on animal behavior. A favorable outcome of these studies will be to increase neuroregeneration following trauma to the CNS, by stimulating either endogenous NS/PC present in the CNS or exogenous populations of NS/PC. This work is pioneering as no similar experiments with lipid blockers have been performed by any other laboratory in any sort of stem cells, nor in the cellular response to neurotrauma and inflammation.

Spinal cord injury. The spinal cords of adult mice are exposed via laminectomy at the level of the 11th and 12th thoracic segments (Goldshmit Y, (2004) J Neurosci 24(45):10064-10073). Briefly, a lumbar spinal left hemisection is performed at the level of the 12th thoracic segment. The survival period post-lesion is 1 and 4 days and 6 weeks. Both SCID mice and mice treated with cyclosporine are used.

Stem cell transplantation. Pre-differentiated NS/PC are injected into the spinal cord at two sites, rostrally and caudally to the injury site. The injections are given at various times following SCI.

Antibody treatment. The anti-LPA mAbs selected from in vitro screening or their isotype controls are injected as follows:

a) at the time of transplantation: either subcutaneously/i.p. or together with the cell transplantation, followed by

b) Every 3 days, after transplantation for 1 day, 4 days or 2 weeks.

Assessment: For anatomical assessment, a animals are sacrificed at various time points (1, 2 and 6 weeks) after cell grafting, and the number of surviving human cells are assessed. Other measurements include neuronal death and/or survival; reactive gliosis; NS/PC responses (proliferation, migration to the injury site and differentiation) and regeneration. Functional assessment consists of locomotion tests. Comparison of efficiency in differentiation, regeneration, neuronal death and glial scarring are performed to assess the efficacy of the combined antibody and stem cell treatment.

### Example 11

### Co-Administration of Anti-LPA Monoclonal Antibody and Adipose-Derived Stem Cells (ASCs) in Experimental Myocardial Infarction

ASCs can be isolated from inguinal subcutaneous adipose tissue according to Danoviz (Danoviz, et al. PLoS One. 2010 Aug. 10; 5(8):e12077) and cultured as described, optionally with the addition of anti-LPA antibody to the cultures to promote survivability prior to inmplantation. Rats can be given experimental myocardial infarction (MI) by surgical ligation of the coronary vasculature (e.g. the LAD). Twenty four hours after MI, animals are given transepicardial injection of ASCs with co-administration of anti-LPA mAb. Animals may also be given anti-LPA antibody treatment systemically (e.g. i.v., s.c., i.p.) to neutralize circulating LPA as well as tissue LPA that would be neutralized with the transepicardial injection. Cardiac function is assessed by echocardiography 30 days post surgery and compared to baseline. Animals are sacrificed for determination of the effects of anti-LPA antibody treatment on the extent of stem cell seeding and transdifferentiation using established markers or by using radiolabeled ASCs.

### Example 12

### Stem Cell-Directed Angiogenesis as a Treatment for the Ischemic Heart

Stem cells have been used to induce therapeutic angiogenesis after myocardial ischemia. Mesenchymal stem cells transplanted in rats after cardial ischemia resulted in enhanced heart function and a small number of the stem cells differentiated into cardiomyocytes. Capillary density was also higher in the stem cell transplanted hearts than in controls. Tang, et al. (2006) Eur. J. Cardio-thoracic Surg. 30:353-361. Using the system of Tang, et al., stem cell transplant is performed in combination with anti-LPA antibody administration. This combination treatment is believed to enhance the success of the stem cell transplant, thus enhancing the efficacy of therapeutic angiogenesis.

Endothelial precursor cells (EPCs) may also be used to promote angiogenesis in the heart after MI or non-MI acute coronary syndrome (ACS). Cardiac ischemia is induced in pigs by surgical coronary ligation, thermocoagulation using a cardiac catheter or by use of an ameroid ring surgically placed around a coronary vessel to promote constriction and subsequent ischemia. EPCs are isolated and cultured in vitro and then allografted into the animals by cardiac catheterization. In combination with EPC delivery, animals are treated with anti-LPA antibody by systemic administration. Seeded EPCs are expected to promote neovascularization to improve blood flow, and the efficacy of the stem cell therapy, and thus of the neovascularization, is believed to be improved by treatment with anti-LPA antibody.

### Example 13

### Beta Cell Regeneration for the Treatment of Diabetes

The challenges and successes of stem cell therapy for Type 1 diabetics have recently been reviewed (Aguaye-Mazzucato, et al., Nat Rev Endocrinol. 2010 March; 6(3):139-48). As is commonly the case for stem cell therapy, conversion of precursor cells into glucose-induced insulin-producing islet cells has not yet been perfected and new strategies are needed. One such new strategy proposed herein is the co-administration of anti-LPA antibodies to enhance stem cell efficacy, e.g., by promotion of transdifferentiation. The preferred animal model for Type 1 diabetes is the STZ rat in which streptozotocin treatment results in the death of islet beta cells and the development of glucose intolerance. Islet precursor cells have been used in this model to restore islet cell function (Li et al Acta Pharmacol Sin. 2010 November; 31(11):1454-63. Epub 2010 Oct. 18). Combination treatment with antibody to LPA can be added to this model and it is believed that this combination will yield enhanced efficacy of islet cell therapy.

All of the compositions and methods described and claimed herein can be made and executed without undue experimentation in light of the present disclosure. While the compositions and methods of this invention have been described in terms of preferred embodiments, it will be apparent to those of skill in the art that variations may be applied to the compositions and methods. All such similar substitutes and modifications apparent to those skilled in the art are deemed to be within the spirit and scope of the invention as defined by the appended claims.

All patents, patent applications, and publications mentioned in the specification are indicative of the levels of those of ordinary skill in the art to which the invention pertains. All patents, patent applications, and publications, including those to which priority or another benefit is claimed, are herein incorporated by reference to the same extent as if each individual publication was specifically and individually indicated to be incorporated by reference.

The invention illustratively described herein suitably may be practiced in the absence of any element(s) not specifically disclosed herein. Thus, for example, in each instance herein any of the terms “comprising”, “consisting essentially of”, and “consisting of” may be replaced with either of the other two terms. The terms and expressions which have been employed are used as terms of description and not of limitation, and there is no intention that in the use of such terms and expressions of excluding any equivalents of the features shown and described or portions thereof, but it is recognized that various modifications are possible within the scope of the invention claimed. Thus, it should be understood that although the present invention has been specifically disclosed by preferred embodiments and optional features, modification and variation of the concepts herein disclosed may be resorted to by those skilled in the art, and that such modifications and variations are considered to be within the scope of this invention as defined by the appended claims.

We claim:

1. A method of stem cell therapy, comprising delivering an inhibitor of lysophosphatidic acid to a stem cell therapy subject during a stem cell therapeutic window, thereby effecting stem cell therapy.

2. The method of claim 1 wherein the inhibitor of lysophosphatidic acid is a direct inhibitor of lysophosphatidic acid.

3. The method of claim 2 wherein the direct inhibitor of lysophosphatidic acid is an agent that binds and neutralizes lysophosphatidic acid.

4. The method of claim 3 wherein the agent that binds and neutralizes lysophosphatidic acid is an antibody, antibody fragment, antibody variant, aptamer, receptor fragment or receptor decoy.

5. The method of claim 4 wherein the antibody, antibody fragment or antibody variant is a monoclonal antibody, or fragment or variant thereof, that binds and neutralizes lysophosphatidic acid.

6. The method of claim 5 wherein the monoclonal antibody, or fragment or variant thereof is a humanized monoclonal antibody or fragment or variant thereof.

7. The method of claim 1 wherein the inhibitor of lysophosphatidic acid is an indirect inhibitor of lysophosphatidic acid.

8. The method of claim 7 wherein the indirect inhibitor of lysophosphatidic acid is a lysophosphatidic acid receptor antagonist, an inhibitor of lysophosphatidic acid biosynthesis, a lysophosphatidic acid-degrading enzyme or an activator or agonist of a lysophosphatidic acid-degrading enzyme.

9. The method of claim 8 wherein the inhibitor of lysophosphatidic acid biosynthesis is an autotaxin inhibitor.

10. A method of preparing stem cells for use in stem cell therapy, comprising culturing said stem cells in the presence of an inhibitor of lysophosphatidic acid.
